(19) United States
(12) Patent Application Publication
(10) Pub. No.: US 2022/0169603 A1
(43) Pub. Date: Jun. 2, 2022

(54) NOVEL SALTS, CRYSTALLINE FORMS AND PREMIX OF HYPOLIPIDEMIC AGENT

(71) Applicant: Cadila Healthcare Ltd., Ahmedabad, Gujarat (IN)

(72) Inventors: Rajiv Sharma, Ahmedabad, Gujarat (IN); Sanjay Kumar, Ahmedabad, Gujarat (IN); Brijesh Kumar Srivastava, Ahmedabad, Gujarat (IN); Harikishore Pingali, Ahmedabad, Gujarat (IN); Ranjeet Nair, Ahmedabad, Gujarat (IN); Kalpesh Shah, Ahmedabad, Gujarat (IN); Pandurang Zaware, Ahmedabad, Gujarat (IN); Manoj Patel, Ahmedabad, Gujarat (IN); Kamal Dave, Ahmedabad, Gujarat (IN)

(21) Appl. No.: 17/437,160

(22) PCT Filed: Mar. 11, 2020
(86) PCT No.: PCT/IB2020/052115

§ 371 Date: Sep. 8, 2021

(30) Foreign Application Priority Data
Mar. 11, 2019 (IN) 201921009322
May 22, 2019 (IN) 201921020258
May 24, 2019 (IN) 201921020611
Sep. 5, 2019 (IN) 201921035730

(51) Int. Cl. C07D 207/333

(52) U.S. Cl. CPC C07D 207/333; C07B 2200/13

(57) ABSTRACT

Present invention relates to novel salts of saroglitazar and certain polymorphic forms of saroglitazar salts. Invention provides process for the preparation of novel salts and polymorphic forms of saroglitazar. Invention also provides novel co-precipitants or premixes of saroglitazar with pharmaceutically acceptable excipients/secondary therapeutic agent and process for the preparing same.

### FIELD OF THE INVENTION

The present invention relates to certain at least partially crystalline forms of salts of saroglitazar of formula (I), processes for the preparation of these novel crystalline and amorphous form of salts, use thereof and pharmaceutical composition comprising the same. The present invention relates to salts of saroglitazar of formula (I), process for the preparation of these novel salts and pharmaceutical composition containing the said salts. Further, the present invention also relates to stable co-precipitates and premixes of Saroglitazar free acid or pharmaceutically acceptable salts of Saroglitazar with pharmaceutically acceptable excipients, methods for their preparation, pharmaceutical compositions containing them and method of using them for treatment thereof.

### BACKGROUND OF THE INVENTION

Hyperlipidemia has been recognized as the major risk factor in causing cardiovascular diseases due to atherosclerosis. Atherosclerosis and other such peripheral vascular diseases affect the quality of life of a large population in the world. The therapy aims to lower the elevated plasma LDL cholesterol, low-density lipoprotein and plasma triglycerides in order to prevent or reduce the risk of occurrence of cardiovascular diseases.

Hypolipidemic agents which are PPAR modulators have been disclosed in WO 91/19702, WO 94/01420, WO 94/13650, WO 95/03038, WO 95/17394, WO 96/04260, WO 96/04261, WO 96/33998, WO 97/25042, WO 97/36579, WO 98/28534, WO 99/08501, WO 99/16758, WO 99/19313, WO99/20614, WO 00/23417, WO 00/23445, WO 00/23451, WO 01/53257.

WO 03009841 discloses compounds of the following general formula:

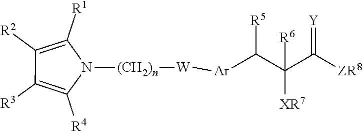

These compounds are reported to be hypolipidemic agents also include Saroglitazar of formula (I).

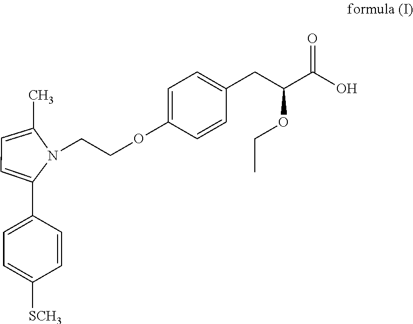 [SMILES: CCO[C@@H](Cc1ccc(OCCn2c(C)ccc2-c2ccc(SC)cc2)cc1)C(=O)O]

WO 03009841 also discloses sodium and calcium salts of saroglitazar. However, these salts were either difficult to isolate due to rapid degradation or were poorly absorbed limiting its efficacy and possibility of further development or were found to degrade on long term storage limiting their suitability for further pharmaceutical development. It has surprisingly now been found that certain crystalline and amorphous form of saroglitazar salts are more stable.

These crystalline and amorphous form of saroglitazar salts can show certain superior pharmaceutical &/or chemical properties.

The saroglitazar of formula (I) is a thick liquid which is difficult to isolate, purify and develop into a pharmaceutical formulation. It is therefore necessary to isolate the acid in a form that is easy to purify, handle, scale up and develop into suitable pharmaceutical formulation. Conversion into suitable salts represents one such means. Thus there is a continuing need to obtain new salts of formula (I) having improved physical and/or chemical properties. The present invention satisfies this need by providing new salts of formula (I).

Certain salts of compound of formula (I) are disclosed in WO2015001573, WO2014195967 and WO2015029066.

Further the Saroglitazar free acid as disclosed in WO03009841 was always obtained in liquid form. When its stability studies were conducted, it was found to be less suitable for further development due to its purity as well as stability concerns as shown below tabular form:

| Saroglitazar | | | |
|---|---|---|---|
| free acid | | | |
| compound- | INITIAL PURITY | | |
| (compound of | and % of impurity | AFTER | AFTER |
| formula I) | (RT-~22.0 min) at 0-5° C. | 6 DAYS | 17 DAYS |
| | 98.11% | 98.44% | 98.39 |
| | (0.573%) | (0.598%) | (0.631%) |

| Free acid | INITIAL PURITY | | |
|---|---|---|---|
| compound- | and | | |
| (compound of | % of impurity | AFTER | AFTER |
| formula I) | (RT-~22.0 min) at 25~30° C. | 6 DAYS | 17 DAYS |
| | 98.11% | 98.12% | 97.87 |
| | (0.573%) | (0.770%) | (0.850%) |

The experimental data in above table shows there is a rise in impurity after 6 and 17 days for the Saroglitazar free acid (compound of formula I). Further, the saroglitazar free acid was found to be less stable at room temperature conditions and hence could not be developed further. Hence there is unmet need for additional solid stable salts of saroglitazar free acid or saroglitazar pharmaceutically acceptable salts.

Salts of compound of formula (I) can be represented by compound of formula (Ia) as shown below:

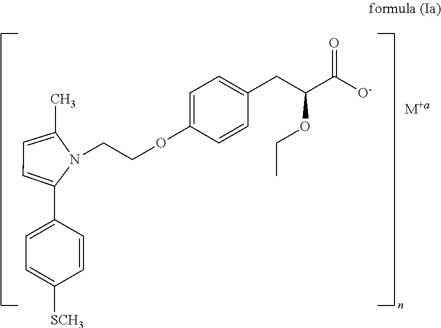

The compound of formula (Ia), wherein M^{+a} is cation, a is valency of cation selected from 1, 2, 3, n is integer selected from 1, 3.

These salts may be present either in substantially crystalline or amorphous forms or may be present as partially crystalline forms. In a preferred embodiment the salts are present in at least partially crystalline form. In another preferred embodiment, the salts are present in an amorphous form. In yet another embodiment the salts are in crystalline form. In another embodiment, the salts are present in non-solvated/unsolvated form or in a solvent free form. In another embodiment, the salts are present in solvated/hydrated form. In yet another embodiment the salts are present in their anhydrous form.

The present invention also discloses certain stable co-precipitates or premix forms of Saroglitazar or pharmaceutically acceptable salt of Saroglitazar with pharmaceutically acceptable excipients. The present invention provides novel premixes and/or co-precipitates of Saroglitazar or its pharmaceutically acceptable salts combined with certain pharmaceutically acceptable excipients.

### OBJECT OF THE INVENTION

In one embodiment of the present invention, there is provided at least partially crystalline form of saroglitazar sodium.

In another embodiment of the present invention, there is provided at least partially crystalline form of saroglitazar magnesium.

In another embodiment of the present invention, there is provided amorphous forms of certain pharmaceutically acceptable salts of saroglitazar, wherein the salt is selected from lithium, potassium and calcium salt of saroglitazar.

In another embodiment of the present invention, there is provided certain new pharmaceutically acceptable salts of saroglitazar.

In yet another embodiment of the present invention, there is provided new co-precipitates or premix form of Saroglitazar or pharmaceutically acceptable salt of saroglitazar with pharmaceutically acceptable excipients or with a secondary therapeutic agent.

In a still further embodiment is provided a pharmaceutical composition comprising, the therapeutically effective amount of different polymorphic forms including the amorphous, partially crystalline and crystalline forms of saroglitazar sodium and saroglitazar magnesium, prepared according to the present invention, along with at least one suitable pharmaceutically acceptable carrier, diluents, vehicle or any other pharmaceutically acceptable any other pharmaceutically acceptable excipient.

In a still further embodiment is provided a pharmaceutical composition comprising, the therapeutically effective amount of different amorphous form of pharmaceutically acceptable salts of saroglitazar, wherein salt is selected from lithium, potassium and calcium, along with at least one suitable pharmaceutically acceptable carrier, diluents, vehicle or any other pharmaceutically acceptable excipient.

In a still further embodiment is provided a pharmaceutical composition comprising, certain novel salts of saroglitazar formula (I), prepared according to the present invention, along with at least one suitable pharmaceutically acceptable carrier, diluents, vehicle or any other pharmaceutically acceptable excipient.

In a still further embodiment is provided a pharmaceutical composition comprising, new co-precipitates or premix form of saroglitazar or pharmaceutically acceptable salt of saroglitazar, prepared according to the present invention, along with at least one suitable pharmaceutically acceptable carrier, diluents, vehicle or along with at leak one suitable pharmaceutically acceptable carrier, diluents, vehicle or any other pharmaceutically acceptable excipient.

Use of different amorphous, partially crystalline and crystalline salts of saroglitazar sodium and saroglitazar magnesium, different amorphous form of pharmaceutically acceptable salts of saroglitazar, wherein salt is selected from lithium, potassium and calcium, certain novel salts of Saroglitazar formula (I), new co-precipitates or premix form of saroglitazar or pharmaceutically acceptable salt of saroglitazar for the treatment of dyslipidemia or hyperglycemia.

### BRIEF DESCRIPTION OF THE DRAWINGS

FIG. 1 is a powder X-ray diffraction (XRPD) pattern of amorphous form of (S) 2-Ethoxy-3-(4-{2-[2-methyl-5-(4-methylthiophenyl)-pyrrol-1-yl]-ethoxy}-phenyl)-propionic acid lithium salt prepared according to Example 1.

FIG. 2 is a powder X-ray diffraction (XRPD) pattern of amorphous form of (S) 2-Ethoxy-3-(4-{2-[2-methyl-5-(4-methylthiophenyl)-pyrrol-1-yl]-ethoxy}-phenyl)-propionic acid potassium salt prepared according to Example 2.

FIG. 3 is a powder X-ray diffraction (XRPD) pattern of amorphous form of (S) 2-Ethoxy-3-(4-{2-[2-methyl-5-(4-methylthiophenyl)-pyrrol-1-yl]-ethoxy}-phenyl)-propionic acid calcium salt prepared according to Example 3.

FIG. 4 is a powder X-ray diffraction (XRPD) pattern of crystalline form of (S) 2-Ethoxy-3-(4-{2-[2-methyl-5-(4-methylthiophenyl)-pyrrol-1-yl]-ethoxy}-phenyl)-propionic acid sodium salt prepared according to Example 4.

FIG. 5 is a powder X-ray diffraction (XRPD) pattern of crystalline form of (S) 2-Ethoxy-3-(4-{2-[2-methyl-5-(4-methylthiophenyl)-pyrrol-1-yl]ethoxy}-phenyl)-propionic acid sodium salt prepared according to Example 5.

FIG. 6 is a powder X-ray diffraction (XRPD) pattern of crystalline form of (S) 2-Ethoxy-3-(4-{2-[2-methyl-5-(4-methylthiophenyl)-pyrrol-1-yl]-ethoxy}-phenyl)-propionic acid sodium salt prepared according to Example 6.

FIG. 7 is a powder X-ray diffraction (XRPD) pattern of crystalline form of (S) 2-Ethoxy-3-(4-{2-[2-methyl-5-(4-methylthiophenyl)-pyrrol-1-yl]-ethoxy}-phenyl)-propionic acid magnesium salt prepared according to Example 7.

FIG. 8 is a powder X-ray diffraction (XRPD) pattern of crystalline form of (S) 2-Ethoxy-3-(4-{2-[2-methyl-5-(4-methylthiophenyl)-pyrrol-1-yl]-ethoxy}-phenyl)-propionic acid magnesium salt prepared according to Example 8.

FIG. 9 is a powder X-ray diffraction (XRPD) pattern of crystalline form of (S) 2-Ethoxy-3-(4-{2-[2-methyl-5-(4-methylthiophenyl)-pyrrol-1-yl]-ethoxy}-phenyl)-propionic acid magnesium salt prepared according to Example 9.

FIG. 10 is a powder X-ray diffraction (XRPD) pattern of amorphous form of (S) 2-Ethoxy-3-(4-{2-[2-methyl-5-(4-methylthiophenyl)-pyrrol-1-yl]-ethoxy}-phenyl)-propionic acid magnesium salt prepared according to Example 10.

FIG. 11 is a powder X-ray diffraction (XRPD) pattern of crystalline form of Metformin-1-glutamic acid-(S)-2-ethoxy-3-(4-(2-(2-methyl-5-(4-(methylthio)phenyl)-1H-pyrrol-1-yl)ethoxy)phenyl)propanoic acid salt prepared according to Example 11.

FIG. 12 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid lithium salt prepared according to Example 12.

FIG. 13 is a powder X-ray diffraction (PXRD) pattern of an amorphous form (S) 2-Ethoxy-3-(4-{2-[2-methyl-5-(4-methylthiophenyl)-pyrrol-1-yl ethoxy phenyl propionic acid potassium salt prepared according to Example 13.

FIG. 14 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid aluminum salt prepared according to Example 14.

FIG. 15 a is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid calcium salt prepared according to Example 16.

FIG. 16 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid calcium salt prepared according to Example 17.

FIG. 17 is a powder X-ray diffraction (PXRD) pattern of amorphous form of (S) 2-Ethoxy-3-(4-{2-[2-methyl-5-(4-methylthiophenyl)-pyrrol-1-yl ethoxy phenyl propionic acid calcium salt prepared according to Example 18.

FIG. 18 is a powder X-ray diffraction (PXRD) pattern of partially crystalline form of (S)-2-ethoxy-3-(4-(2-(2-methyl-5-(4-(methylthio)phenyl)-1H-pyrrol-1-yl)ethoxy)phenyl)propanoic acid sodium salt prepare according to Example 19.

FIG. 19 is a powder X-ray diffraction (PXRD) pattern of partially crystalline form of (S)-2-ethoxy-3-(4-(2-methyl-5-(4-(methylthio)phenyl)-1H-pyrrol-1-yl)ethoxy)phenyl)propanoic acid sodium salt prepared according to Example 20.

FIG. 20 is a powder X-ray diffraction (PXRD) pattern of partially crystalline form of (S)-2-ethoxy-3-(4-(2-(2-methyl-5-(4-(methylthio)phenyl)-1H-pyrrol-1-yl)ethoxy)phenyl)propanoic acid sodium salt prepared according to Example 21.

FIG. 21 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid sodium salt prepared according to Example 22.

FIG. 22 is a powder X-ray diffraction (PXRD) pattern of partially crystalline form of (S)-2-ethoxy-3-(4-(2-(2-methyl-5-(4-(methylthio)phenyl)-1H-pyrrol-1-yl)ethoxy)phenyl)propanoic acid sodium salt prepared according to Example 23.

FIG. 23 is a powder X-ray diffraction (PXRD) pattern of partially crystalline form of (S)-2-ethoxy-3-(4-(2-(2-methyl-5-(4-(methythio)phenyl)-1H-pyrrol-1-yl)ethoxy)phenyl)propanoic acid sodium salt prepared according to Example 24.

FIG. 24 is a powder X-ray diffraction (PXRD) pattern of crystalline form of (S)-2-ethoxy-3-(4-(2-(2-methyl-5-(4-(methylthio)phenyl)-1H-pyrrol-1-yl)ethoxy)pheny)propanoic acid sodium salt prepared according to Example 25.

FIG. 25 is a powder X-ray diffraction (PXRD) pattern of an amorphous form of (S) 2-Ethoxy-3-(4-(2-methyl-5-(4-methylthio)phenyl)-1H-pyrrol-1-yl) ethoxy)phenyl) propionic acid sodium salt prepared according to Example 26.

FIG. 26 is a powder X-ray diffraction (PXRD) pattern of partially crystalline form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 27.

FIG. 27 is a powder X-ray diffraction (PXRD) pattern of partially crystalline form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 28.

FIG. 28 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 29.

FIG. 29 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 30.

FIG. 30 is a powder X-ray diffraction (PXRD) pattern of a partially crystalline form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 31.

FIG. 31 is a powder X-ray diffraction (PXRD) pattern of a partially crystalline form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 32.

FIG. 32 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 33.

FIG. 33 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 34.

FIG. 34 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 35.

FIG. 35 is a powder X-ray diffraction (PXRD) pattern of an amorphous form of (S)-2-ethoxy-3-(4-(2-(2-methyl-5-(4-(methylthio)phenyl)-1H-pyrrol-1-yl)ethoxy)phenyl)propanoic acid magnesium salt prepared according to Example 36.

FIG. 36 is a powder X-ray diffraction (PXRD) pattern of amorphous Co-precipitate or premix of Saroglitazar free acid premixed with copovidone (1:2) obtained according to the example 43.

FIG. 37 is a powder X-ray diffraction (PXRD) pattern of amorphous Co-precipitate or premix of saroglitazar magnesium with HPMC (1:2) obtained according to the example 44.

FIG. 38 is a powder X-ray diffraction (PXRD) pattern of amorphous Co-precipitate or premix of saroglitazar magnesium with HPMC (1:3) obtained according to the example 45.

FIG. 39 is a powder X-ray diffraction (PXRD) pattern of amorphous Co-precipitate or premix of saroglitazar magnesium with HPMC (1:4) obtained according to the example 46.

FIG. 40 is a powder X-ray diffraction (PXRD) pattern of amorphous Co-precipitate or premix of saroglitazar magnesium with HPMC (1:5) obtained according to the example 47.

FIG. 41 is a powder X-ray diffraction (PXRD) pattern of amorphous Co-precipitate or premix of saroglitazar magnesium with methacrylic acid (Eudragit) (1:2) obtained according to the example 48.

FIG. 42 is a powder X-ray diffraction (PXRD) pattern of amorphous Co-precipitate or premix of saroglitazar magnesium with methacrylic acid (Eudragit) (1:3) obtained according to the example 49.

FIG. 43 is a powder X-ray diffraction (PXRD) pattern of amorphous Co-precipitate or premix of saroglitazar magnesium with methacrylic acid (Eudragit) (1:4) obtained according to the example 50.

FIG. 44 is a powder X-ray diffraction (PXRD) pattern of amorphous Co-precipitate or premix of saroglitazar magnesium with methacrylic acid (Eudragit) (1:5) obtained according to the example 51.

FIG. 45 is a powder X-ray diffraction (PXRD) pattern of crystalline a of Saroglitazar free acid with metformin obtained according to the example 52.

FIG. 46 is a powder X-ray diffraction (PXRD) pattern of amorphous Co-precipitate or premix of Saroglitazar free acid premixed with magnesium aluminometasilicate obtained according to the example 53.

FIG. 47 is a powder X-ray diffraction (XRPD) pattern of partially crystalline form of (S) 2-Ethoxy-3-(4-{2-[2-methyl-5-(4-methylthiophenyl)-pyrrol-1-yl]-ethoxy}-phenyl)-propionic acid Magnesium salt prepare according to the example 71.

FIG. 48 is a powder X-ray diffraction (XRPD) pattern of partially crystalline form of (S) 2-Ethoxy-3-(4-{2-[2-methyl-5-(4-methylthiophenyl)-pyrrol-1-yl]-ethoxy}-phenyl)-propionic acid Magnesium salt prepare according to the example 72.

### ABBREVIATIONS

DCM: Dichloromethane
Eudragit: Methacrylic acid
GC: Gas chromatography
HPLC: High-performance liquid chromatography
HPMC: Hydroxypropyl Methylcellulose
HPMC.AS: Hydroxypropyl methylcellulose acetate succinate
HCl: Hydrochloric acid
MeOH: Methanol
Neusilin: Magnesium aluminometasilicate
PXRD: Powder X-ray diffraction
RT: Room Temperature
UPLC: Ultra-performance liquid chromatography

### DETAILED DESCRIPTION OF THE INVENTION

As herein used the term saroglitazar or saroglitazar free acid or (S) 2-Ethoxy-3-(4-{2-[2-methyl-5-(4-methylthiophenyl)-pyrrol-1-yl]-ethoxy}-phenyl)-propionic acid or formula (I) in acid form is the compound having the following formula

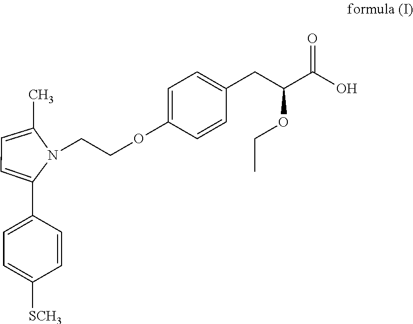 [SMILES: CCO[C@@H](Cc1ccc(OCCn2c(C)ccc2-c2ccc(SC)cc2)cc1)C(=O)O]

The term salt of saroglitazar or saroglitazar salt used anywhere in the specification means a cation along with saroglitazar acid. It is represented by formula (Ia), wherein M^{+a} is a cation; ‘a’ is the valency of the cation and is selected from 1, 2, 3; n is an integer selected from 1, 2, 3. M^{+a} is selected from Cesium (Cs), Copper (Cu), Cobalt (Co), Iron (Fe), Manganese (Mn) and Lead (Pb), Aluminum (Al). M^{+a} also represents metformin or metformin-1-glutamic acid ions.

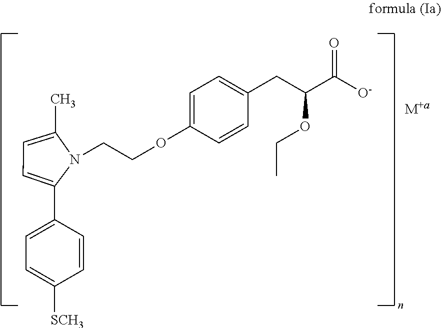

The term ‘premix’ as used herein refers to a solid composition, generally powders or granules, of Saroglitazar free acid or its pharmaceutically acceptable salts with least one pharmaceutically excipient or secondary therapeutic agent.

The terms ‘co-precipitate’ as used in the present invention are synonymous and are intended to mean a dispersion of Saroglitazar or pharmaceutically acceptable salt of Saroglitazar in an inert carrier or matrix in a solid state prepared by dissolving Saroglitazar or pharmaceutically acceptable salt of Saroglitazar carboxylic acid and one or more pharmaceutical excipients in a solvent or solvent mixture and removing the solvent or solvent mixture.

In one embodiment of the present invention, there is provided certain forms of Saroglitazar sodium which are in at least partially crystalline form.

In an embodiment the sodium salts may be present either in partially crystalline forms, crystalline form or amorphous form.

In an embodiment, at least partially crystalline saroglitazar sodium is having crystalline purity in the range of at least 10-90%, preferably crystalline purity 10-80%, or crystalline purity of at least 10-70%, or crystalline purity of at least 10-60%, or crystalline purity of at least 10-50%, or crystalline purity of at least 10-40%, or crystalline purity of at least 10-30%, or crystalline purity of at least 10-20% and including all values in between the defined ranges.

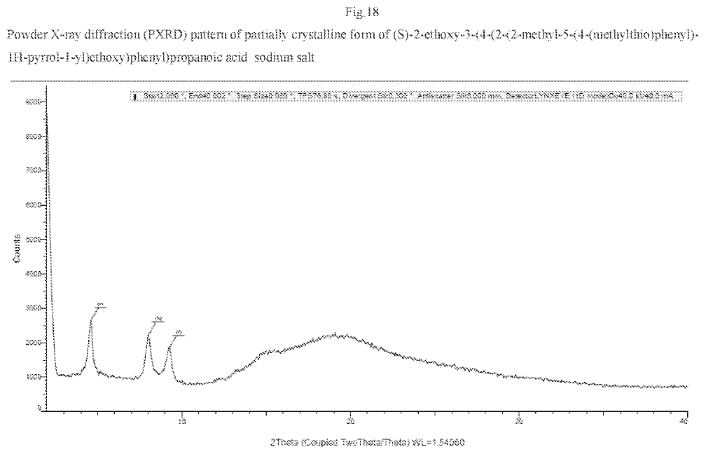

In an embodiment of the present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid sodium salt having at least one of the following characteristics:
i) A partially crystalline amorphous form of (S)-2-ethoxy-3-(4-(2-(2-methyl-5-(4-(methylthio)phenyl)-1H-pyrrol-1-yl)ethoxy)phenyl)propanoic acid sodium salt having a PXRD pattern as per FIG. 18;
ii) Powder XRD peaks at 4.567, 8.006, 9.219±0.2 degrees 2-theta;
iii) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having a melting point of 243.8° C.

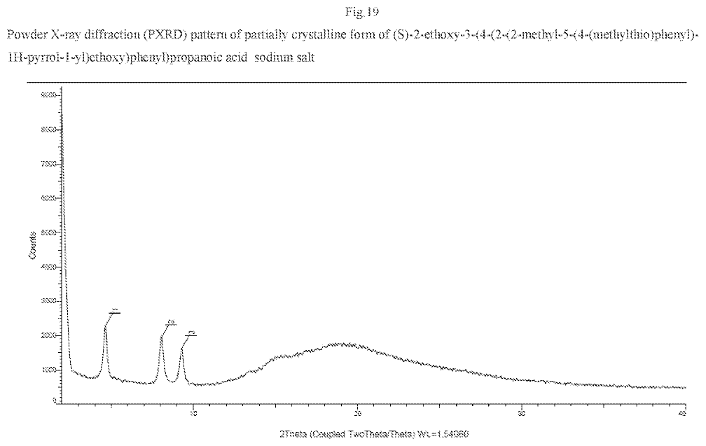

In an embodiment of the present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid sodium salt having at least one of the following characteristics:
i) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt has PXRD pattern as per FIG. 19;
ii) Powder XRD peaks at 4.632, 8.053, 9.286±0.2 degrees 2-theta.

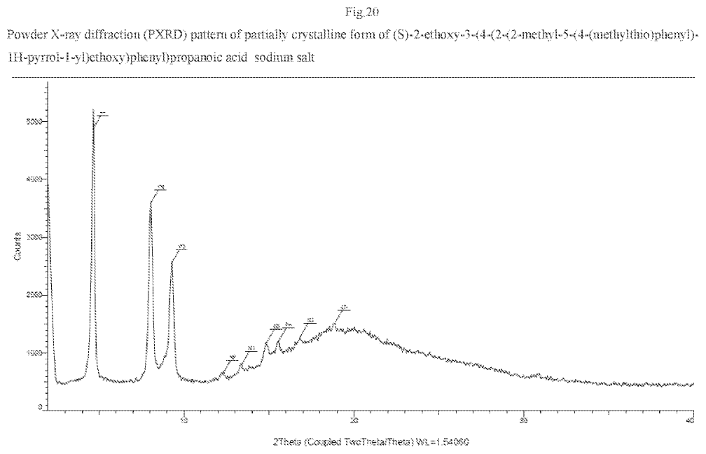

In an embodiment of the present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid sodium salt having at least one of the following characteristics:
i) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having PXRD as per FIG. 20.
ii) Powder XRD peaks at 4.643, 8.044, 9.277, 12.269, 13.381, 14.860±0.2 degrees 2-theta;
iii) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having a melting point of 240.6° C.

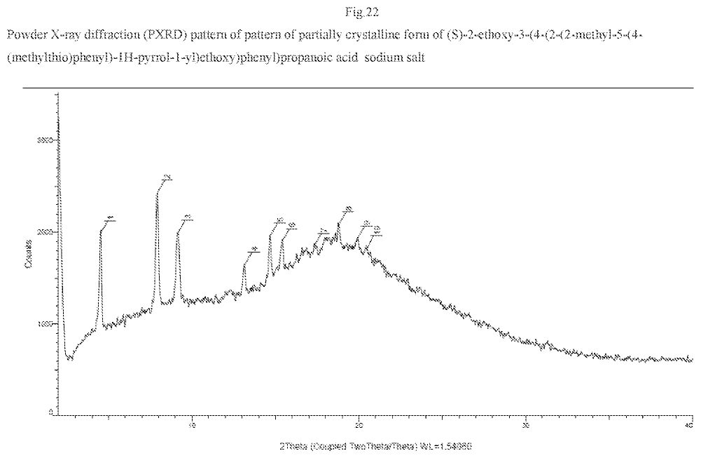

In an embodiment of the present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid sodium salt which havings at least one of the following characteristics:
i) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having PXRD as per FIG. 22.
ii) Powder XRD peaks at 4.513, 7.918, 9.147, 13.129, 14.678, 15.385±0.2 degrees 2-theta;
iii) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having a melting point of 182.4° C.

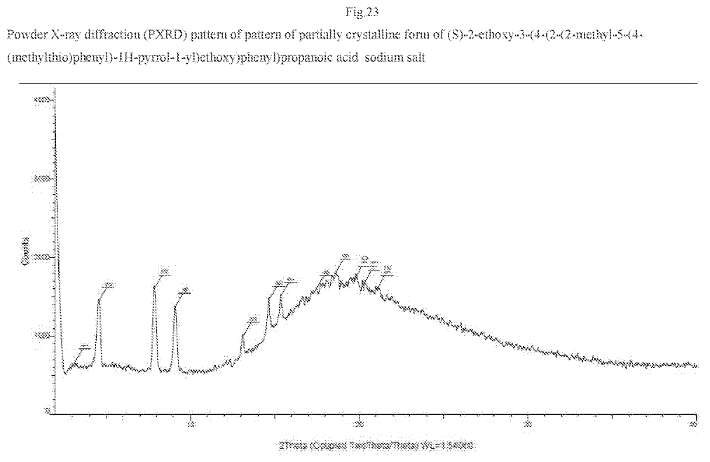

In an embodiment of present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid sodium salt which having at least one of the following characteristics:
i) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having a PXRD as per FIG. 23;
ii) Powder XRD peaks at 3.158, 4.552, 7.873, 9.102, 3.131, 14.655, 5.363, 17.447±0.2 degrees 2-theta;
iii) A partially crystalline form of (S)-2-ethoxy-3-(4-(2-(2-methyl-5-(4-(methylthio)phenyl)-1H-pyrrol-1-yl)ethoxy)phenyl)propanoic acid sodium salt having a melting point of 267.1° C.

In one embodiment of the present invention is provided a crystalline form of (S) 2-Ethoxy-3-(4-{2-[2-methyl-5-(4-methylthiophenyl)-pyrrol-1-yl]-ethoxy}-phenyl)-propionic acid sodium salt.

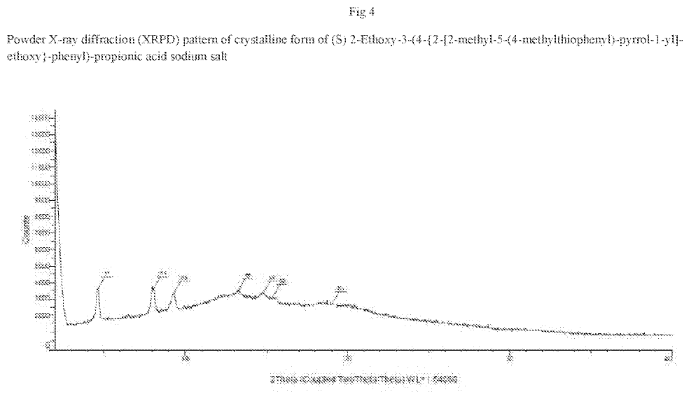

In a further embodiment of the invention disclosed crystalline form of (S) 2-Ethoxy-3-(4-{2-[2-methyl-5-(4-methylthiophenyl)-pyrrol-1-yl]-ethoxy}-phenyl)-propionic acid Sodium salt which has at least one of the following characteristics:
i) A powder X-ray diffraction pattern substantially in accordance with FIG. 4;
ii) A powder X-ray diffraction pattern having peaks at about 4.628, 8.043, 9.288±0.2 degrees 2-theta;
iv) A powder X-ray diffraction pattern having additional peaks at about 13.218, 14.760, 15.381, 19.007±0.2 degrees 2-theta.
v) Crystalline form 1 of (S) 2-Ethoxy-3-(4-{2-[2-methyl-5-(4-methylthiophenyl)-pyrrol-1-yl]-ethoxy}-phenyl)-propionic acid Sodium salt characterized by a melting point 238.3° C.±2° C.

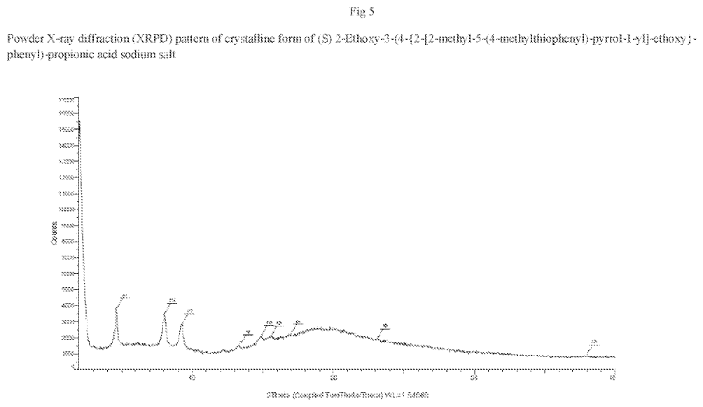

In a further embodiment of the invention disclosed crystalline Form 2 of (S) 2-Ethoxy-3-(4-{2-[2-methyl-5-(4-methylthiophenyl)-pyrrol-1-yl]-ethoxy}-phenyl)-propionic acid Sodium salt which has at least one of the following characteristics:
i) A powder X-ray diffraction pattern substantially in accordance with FIG. 5;
ii) A powder X-ray diffraction pattern having peaks at about 4.605, 8.019, 9.254±0.2 degrees 2-theta;
iii) A powder X-ray diffraction pattern having additional peaks at about 13.324, 14.846, 15.518, 16.919, 23.070, 37.940±0.2 degrees 2-theta.
iv) Crystalline Form 2 of (S) 2-Ethoxy-3-(4-{2-[2-methyl-5-(4-methylthiophenyl)-pyrrol-1-yl]-ethoxy}-phenyl)-propionic acid Sodium salt characterized by a melting point 263.2° C.±2° C.

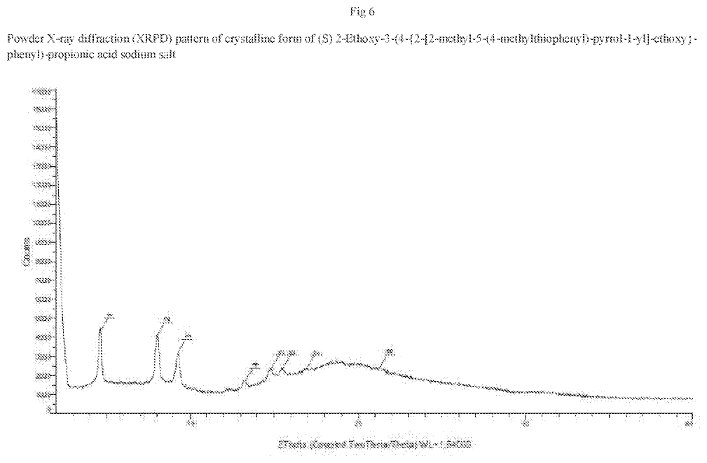

In a further embodiment of the invention disclosed crystalline Form 3 of (S) 2-Ethoxy-3-(4-{2-[2-methyl-5-(4-methylthiophenyl)-pyrrol-1-yl]-ethoxy}-phenyl)-propionic acid sodium salt which has at least one of the following characteristics:
i) A powder X-ray diffraction pattern substantially in accordance with FIG. 6;
ii) A powder X-ray diffraction pattern having peaks at about 4.603, 8.020, 9.257±0.2 degrees 2-theta;
iii) A powder X-ray diffraction pattern having additional peaks at about 13.288, 14.816, 15.502, 16.912, 21.276±0.2 degrees 2-theta.
iv) Crystalline Form 3 of (S) 2-Ethoxy-3-(4-{2-[2-methyl-5-(4-methylthiophenyl)-pyrrol-1-yl]-ethoxy}-phenyl)-propionic acid. Sodium salt characterized by a melting point 240° C.±2° C.

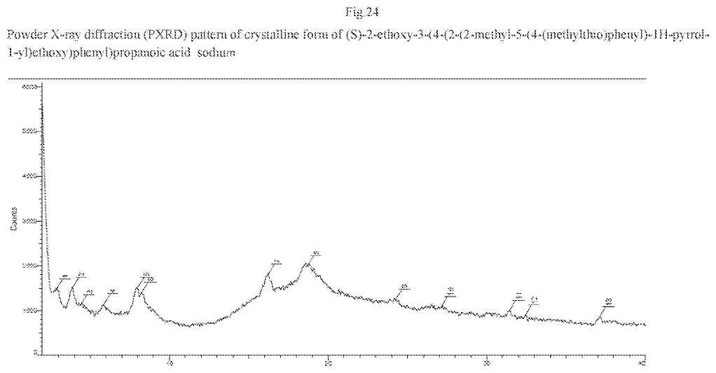

In a further embodiment of the invention disclosed crystalline form of (S) 2-Ethoxy-3-(4-{2-[2-methyl-5-(4-methylthiophenyl)-pyrrol-1-yl]-ethoxy}-phenyl)-propionic acid sodium salt which has at least one of the following characteristics:
i) A powder X-ray diffraction pattern substantially in accordance with FIG. 24;
ii) A powder X-ray diffraction pattern having peaks at about 3.864, 7.952, 8.225, 16.195, 18.663±0.2 degrees 2-theta;

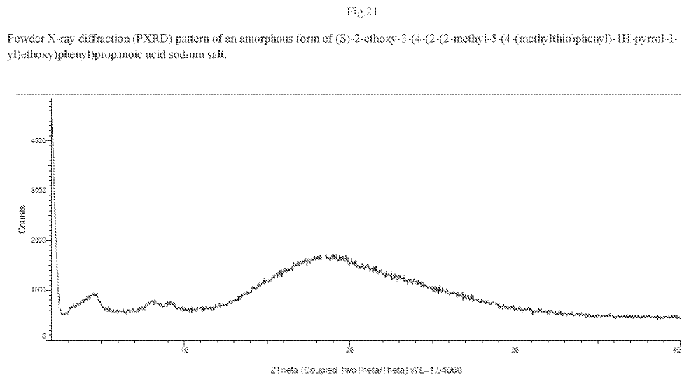

In an embodiment of the present invention is provided an amorphous form of (S)-2-ethoxy-3-(4-(2-(2-methyl-5-(4-(methylthio)phenyl)-1H-pyrrol-1-yl)ethoxy)phenyl)propanoic acid sodium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid sodium salt has PXRD as per FIG. 21.
ii) An amorphous form of (S)-2-ethoxy-3-(4-(2-(2-methyl-5-(4-(methylthio)phenyl)-1H-pyrrol-1-yl)ethoxy)phenyl)propanoic acid sodium salt has melting point 139.1° C.

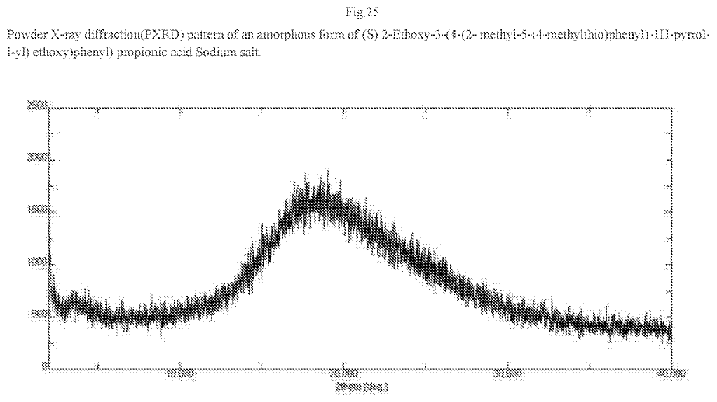

In an embodiment of present invention is provided an amorphous form of (S) 2-Ethoxy-3-(4-(2-methyl-5-(4-methylthio)phenyl)-1H-pyrrol-1-yl)ethoxy)phenyl) propionic acid sodium salt which has at least one of the following characteristics:
i) An amorphous form of (S) 2-Ethoxy-3-(4-(2-methyl-5-(4-methylthio)phenyl)-1H-pyrrol-1-yl)ethoxy)phenyl) propionic acid Sodium salt has PXRD as per FIG. 25.
ii) An amorphous form of (S) 2-Ethoxy-3-(4-(2-methyl-5-(4-methylthio)phenyl)-1H-pyrrol-1-yl) ethoxy)phenyl)propionic acid Sodium salt has melting point 72.6° C.

In one embodiment of the present invention, there is provided certain forms of saroglitazar magnesium which are in at least partially crystalline form.

In an embodiment magnesium salts may be present either in partially crystalline forms, crystalline or amorphous forms.

In an embodiment, at least partially crystalline saroglitazar sodium is having crystalline purity in the range of at least 10-90%, preferably crystalline purity 10-80%, or crystalline purity of at least 10-70%, or crystalline purity of at least 10-60%, or crystalline purity of at least 10-50%, or crystalline purity of at least 10-40%, or crystalline purity of at least 10-30%, or crystalline purity of at least 10-20% and including all values in between the defined ranges.

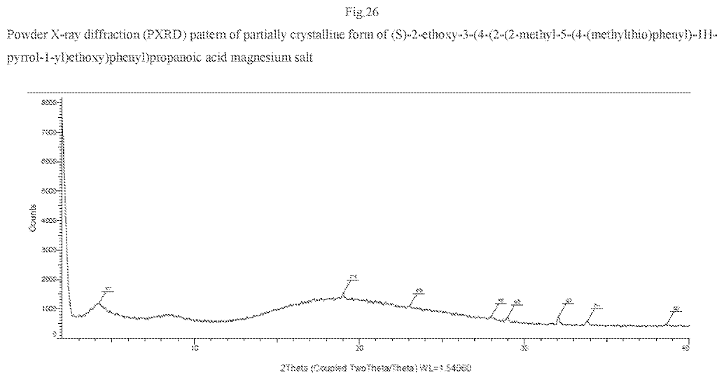

In an embodiment of the present invention is provided a leak partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 26;
ii) Powder XRD peaks at 4.177, 19.029, 23.038, 32.092, 33.818±0.2 degrees 2-theta;
iii) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has a melting point of 171.5° C.

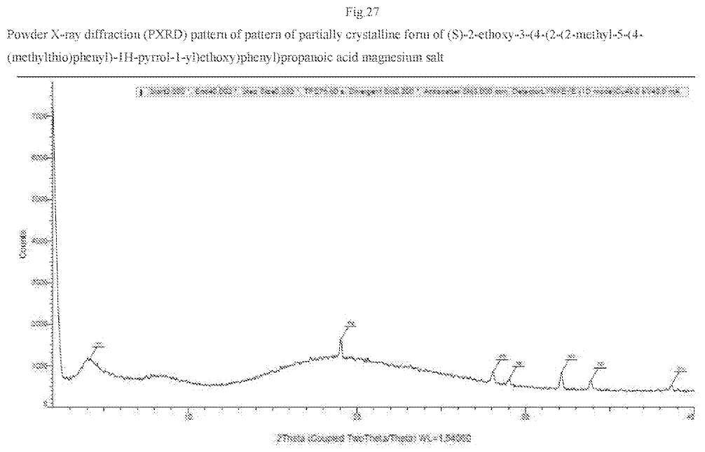

In an embodiment of present invention is provided at least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 27.
iv) Powder XRD peaks at 4.122, 19.033, 28.037, 29.009, 32.121, 33.870±0.2 degrees 2-theta;
ii) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has melting point 179.2° C.

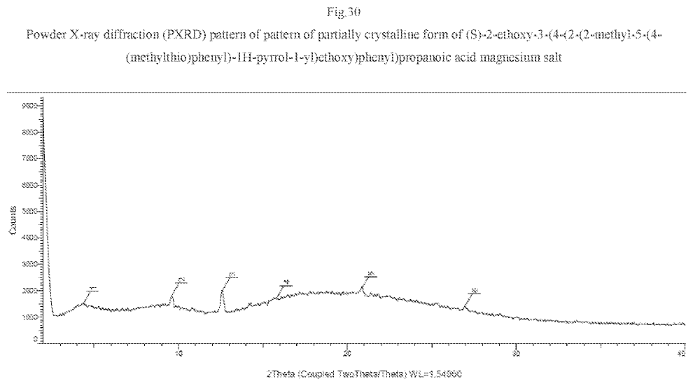

In embodiment of present invention is provided a partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) A partially crystalline form of (S)-2-ethoxy-3-(4-(2-(2-methyl-5-(4-(methylthio)phenyl)-1H-pyrrol-1-yl)ethoxy)phenyl)propanoic acid magnesium salt (prepared according to example 31) has PXRD as per FIG. 30;
ii) Powder XRD peaks at 4.346, 9.584, 12.579, 20.803±0.2 degrees 2-theta;
iii) A partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has a melting point 260.7° C.

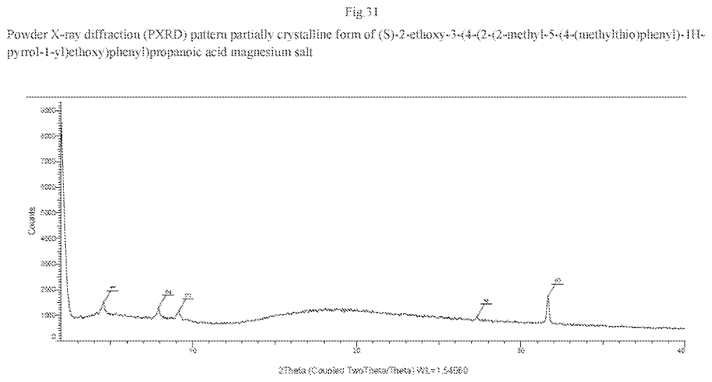

In embodiment of present invention is provided at least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 31.
ii) Powder XRD peaks at 4.538, 7.916, 9.134, 31.650±0.2 degrees 2-theta;
iii) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has melting point 137.0° C.

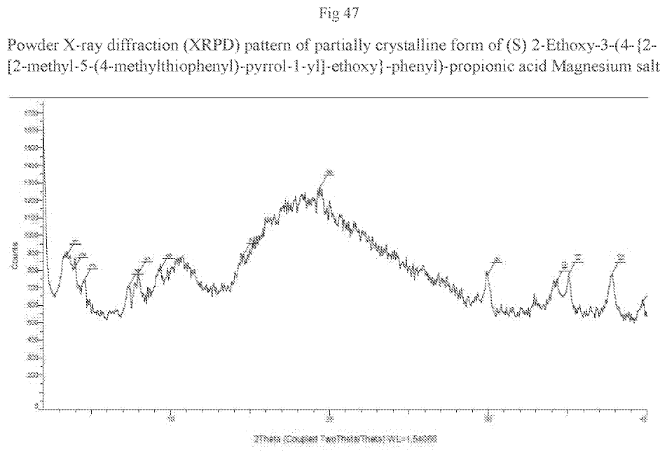

In an embodiment of present invention is provided at least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 47;
ii) Powder XRD peaks at 7.935, 4.533, 29.933, 34.228, 35.057±0.2 degrees 2-theta;
iii) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has melting point 269.8° C.

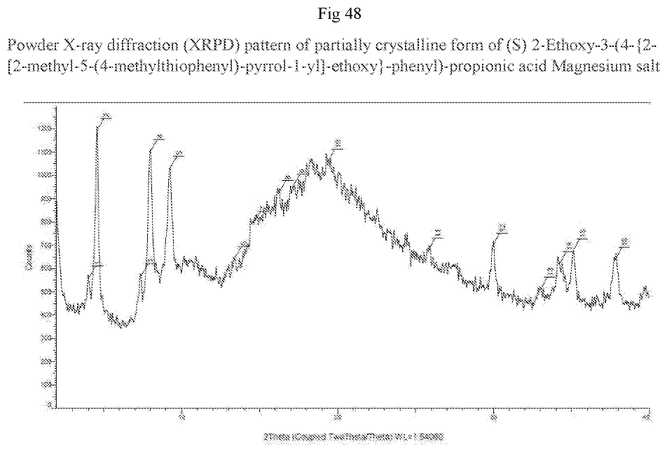

In an embodiment of present invention is provided at least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) At least partially crystalline form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 48;
ii) Powder XRD peaks at 4.572, 7.386, 7.990, 9.248, 29.957, 35.116, 37.802±0.2 degrees 2-theta;

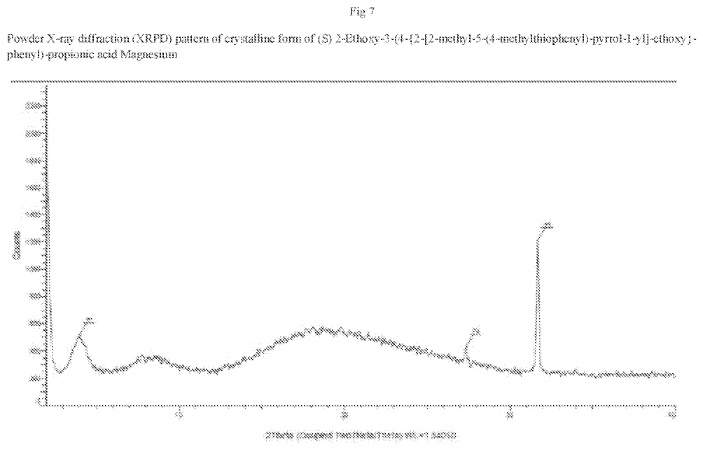

In a further embodiment of the invention disclosed crystalline Form 1 of (S) 2-Ethoxy-3-(4-{2-[2-methyl-5-(4-methylthiophenyl)-pyrrol-1-yl]-ethoxy}-phenyl)-propionic acid magnesium salt which has at least one of the following characteristics:
i) A powder X-ray diffraction pattern substantially in accordance with FIG. 7;
ii) A powder X-ray diffraction pattern having peaks at about 3.980, 27.350, 31.685±0.2 degrees 2-theta;
iii) Crystalline Form 1 of (S) 2-Ethoxy-3-(4-{2-[2-methyl-5-(4-methylthiophenyl)-pyrrol-1-yl]-ethoxy}-phenyl)-propionic acid magnesium salt characterized by a melting point 148.4° C.±2° C.

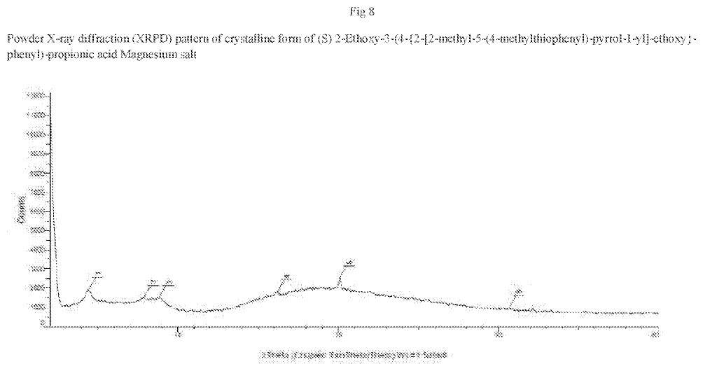

In a further embodiment of the invention disclosed crystalline Form 2 of (S) 2-Ethoxy-3-(4-{2-[2-methyl-5-(4-methylthiophenyl)-pyrrol-1-yl]-ethoxy}-phenyl)-propionic acid magnesium salt, which has at least one of the following characteristics:
i) A powder X-ray diffraction pattern substantially in accordance with FIG. 8;
ii) A powder X-ray diffraction pattern having peaks at about 4.418, 8.809, 20.074±0.2 degrees 2-theta;
iii) A powder X-ray diffraction pattern having additional peaks at about 7.855, 16.165, 30.678±0.2 degrees 2-theta.
iv) Crystalline Form 2 of (S) 2-Ethoxy-3-(4-{2-[2-methyl-5-(4-methylthiophenyl)-pyrrol-1-yl]-ethoxy}-phenyl)-propionic acid Magnesium salt characterized by a melting point 223° C.±2° C.

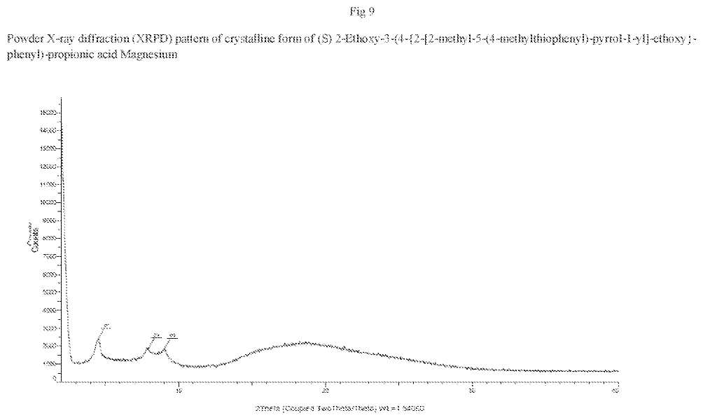

In a further embodiment of the invention disclosed crystalline Form 3 of (S) 2-Ethoxy-3-(4-{2-[2-methyl-5-(4-methylthiophenyl)-pyrrol-1-yl]-ethoxy}-phenyl)-propionic acid magnesium salt which has at least one of the following characteristics:
i) A powder X-ray diffraction pattern substantially in accordance with FIG. 9;
ii) A powder X-ray diffraction pattern having peaks at, 4.488, 7.896, 8.979±0.2 degrees 2-theta;
iii) Crystalline Form 3 of (S) 2-Ethoxy-3-(4-{2-[2-methyl-5-(4-methylthiophenyl)-pyrrol-1-yl]ethoxy}-phenyl)-propionic acid Magnesium salt characterized by a melting point in a range about 185° C.±2° C. to 190° C.±2° C.

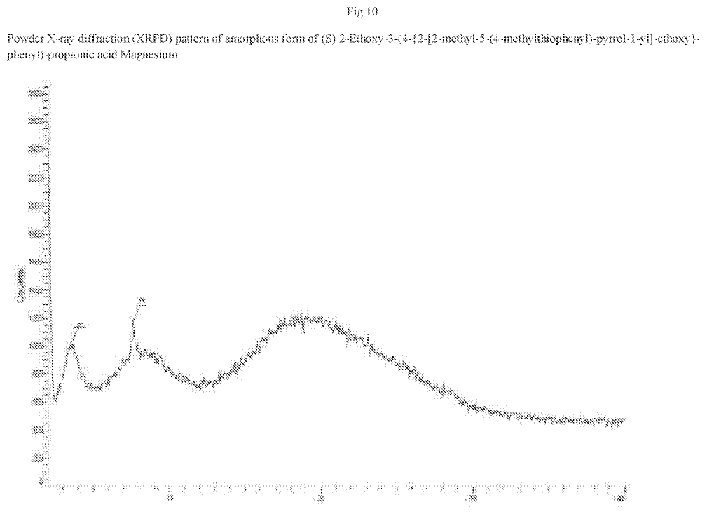

In a further embodiment of the invention is disclosed novel amorphous form of (S) 2-Ethoxy-3-(4-{2-[2-methyl-5-(4-methylthiophenyl)-pyrrol-1-yl]-ethoxy}-phenyl)-propionic acid magnesium salt, which has at least one of the following characteristics:
i) A powder X-ray diffraction pattern substantially in accordance with FIG. 10;
ii) Amorphous of (S) 2-Ethoxy-3-(4-{2-[2-methyl-5-(4-methylthiophenyl)-pyrrol-1-yl]-ethoxy}-phenyl)-propionic acid Magnesium salt characterized by a melting point in the range about 219° C.±2° C.

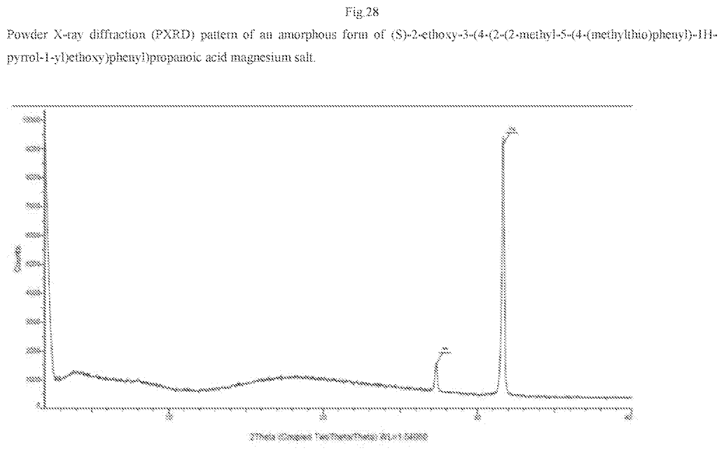

In an embodiment of present invention is provided a novel amorphous form of (S)-2-ethoxy-3-(4-(2-methyl-5-(4-(methylthio)phenyl)-1H-pyrrol-1-yl)ethoxy)phenyl)propanoic acid magnesium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid magnesium salt has PXRD as per FIG. 28;
ii) Powder XRD peaks at 27.322, 31.653±0.2 degrees 2-theta;
iii) An amorphous form of (S)-2-ethoxy-3-(4-(2-(2-methyl-5-(4-(methylthio)phenyl)-1H-pyrrol-1-yl)ethoxy)phenyl)propanoic acid magnesium salt has a melting point of 154° C.

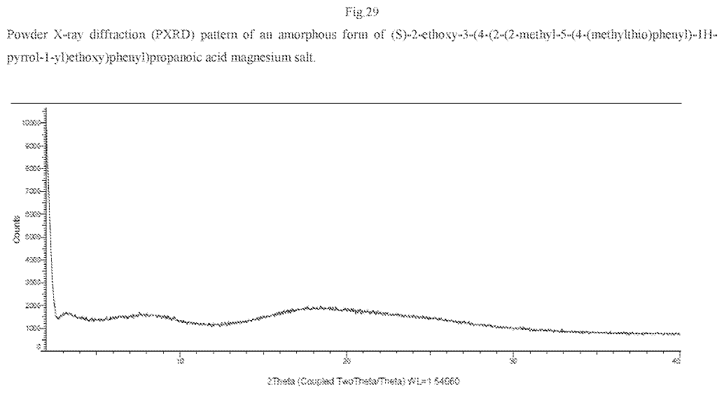

In an embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid magnesium salt having a PXRD pattern as per FIG. 29.

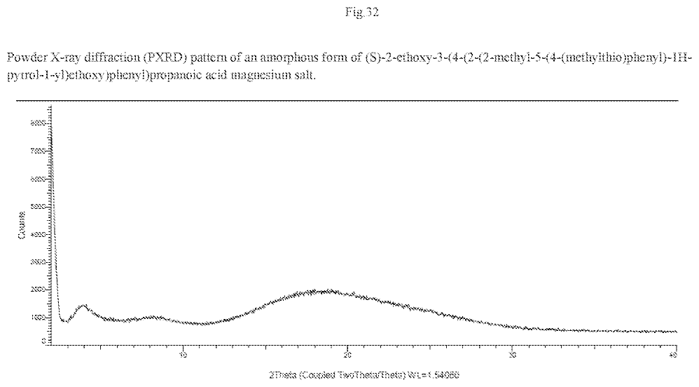

In an embodiment of present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has PXRD pattern as per FIG. 32;
ii) An amorphous form of (S)-2-ethoxy-3-(4-(2-(2-methyl-5-(4-(methylthio)phenyl)-1H-pyrrol-1-yl)ethoxy)phenyl)propanoic acid magnesium salt has melting point 110.4° C.

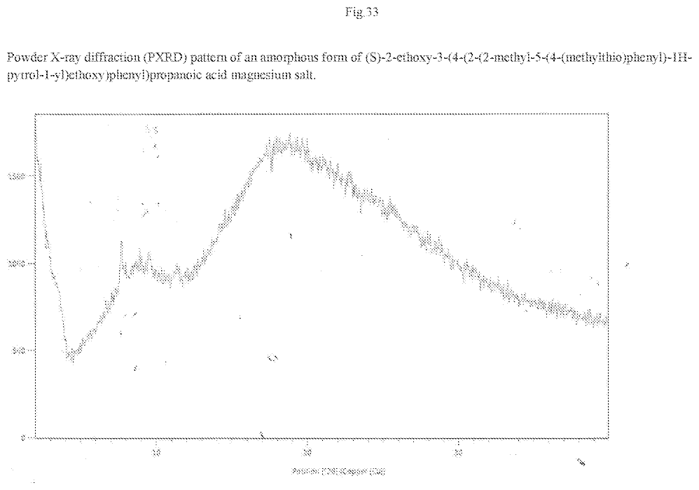

In one embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has PXRD pattern as per FIG. 33;
ii) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has melting point 271.6° C.

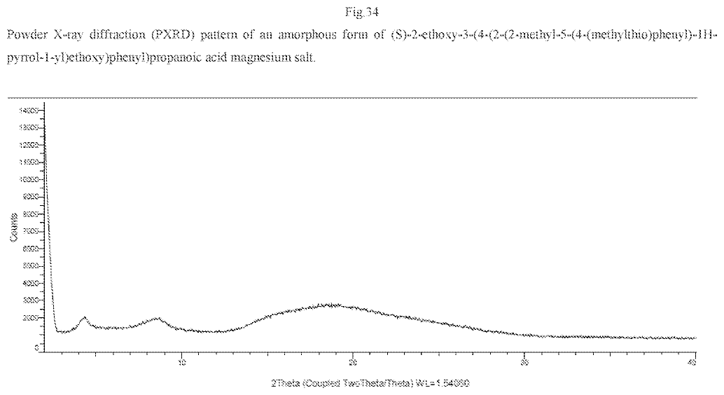

In one embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has PXRD as per FIG. 34;
ii) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has melting point 265.1° C.

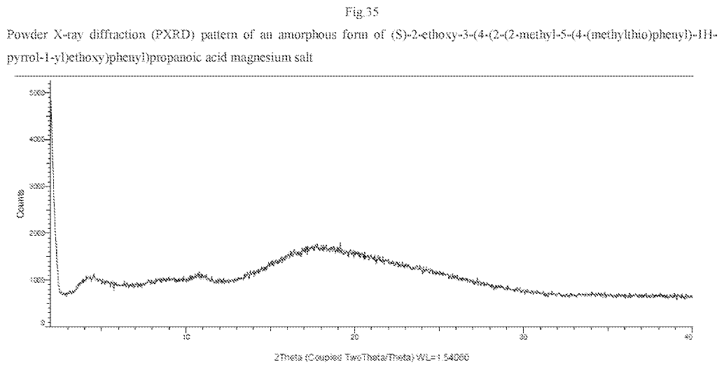

In one embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid magnesium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid magnesium salt which has PXRD pattern as per FIG. 35;

In one embodiment of the present invention, there is provided amorphous forms of certain pharmaceutically acceptable salts of saroglitazar, wherein the salt is selected from lithium, potassium and calcium salts.

In one embodiment of the present invention, there is provided certain amorphous forms of saroglitazar lithium salts.

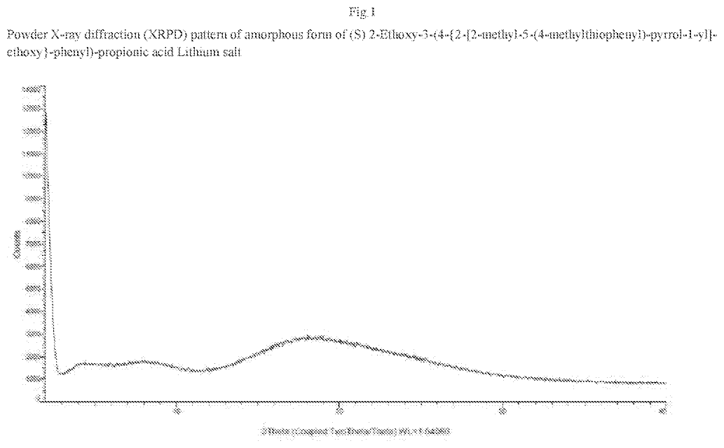

In one embodiment of the present invention is provided a novel amorphous form of (S) 2-Ethoxy-3-(4-{2-[2-methyl-5-(4-methylthiophenyl)-pyrrol-1-yl]-ethoxy}-phenyl)-propionic acid lithium salt which has at least one of the following characteristics:
i) A powder X-ray diffraction pattern substantially in accordance with FIG. 1;
ii) (S) 2-Ethoxy-3-(4-{2-[2-methyl-5-(4-methylthiophenyl)-pyrrol-1-yl]-ethoxy}-phenyl)-propionic acid lithium salt characterized by a melting point 131.2° C.±2° C.

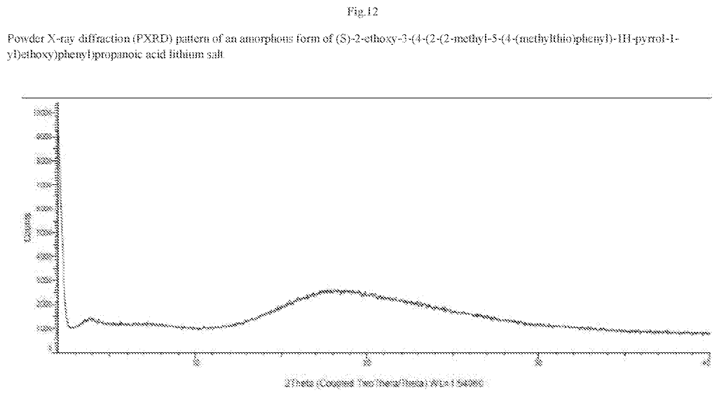

In one embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid lithium salt which has at least one of the following characteristics:
i) a powder X-ray diffraction pattern substantially in accordance with FIG. 12;
ii) (S)-2-ethoxy-3-(4-(2-(2-methyl-5-(4-(methylthio)phenyl)-1H-pyrrol-1-yl)ethoxy)phenyl)propanoic acid lithium salt characterized by a melting point 285.5° C.

In one embodiment of the present invention, there is provided certain novel amorphous forms of saroglitazar potassium salts.

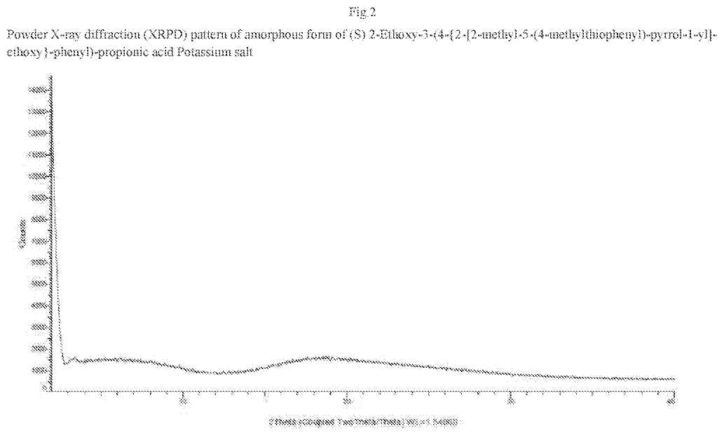

In one embodiment of the present invention is provided a novel amorphous form of (S) 2-Ethoxy-3-(4-{2-[2-methyl-5-(4-methylthiophenyl)-pyrrol-1-yl]-ethoxy}-phenyl)-propionic acid potassium salt which has at least one of the following characteristics:
i) A powder X-ray diffraction pattern substantially in accordance with FIG. 2;
ii) (S) 2-Ethoxy-3-(4-{2-[2-methyl-5-(4-methylthiophenyl)-pyrrol-1-yl]-ethoxy}-phenyl)-propionic acid potassium salt characterized by a melting point 62.1° C.±2° C.

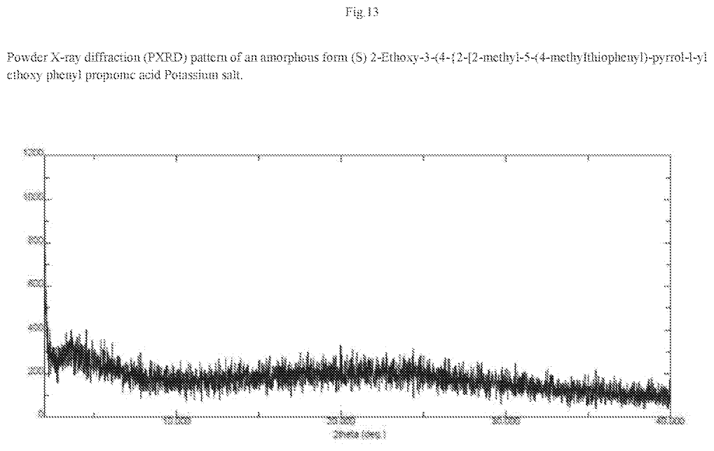

In an embodiment of the present invention is provided a novel amorphous form of (S) 2-Ethoxy-3-(4-{2-[2-methyl-5(4-methylthiophenyl)-pyrrol-1-yl]-ethoxy}-phenyl)-propionic acid potassium salt which has at least one of the following characteristics:
i) Amorphous form of (S) 2-Ethoxy-3-(4-{2-[2-methyl-5-(4-methylthiophenyl)-pyrrol-1-yl]-ethoxy}-phenyl)-propionic acid potassium salt has PXRD pattern as per FIG. 13.

In one embodiment of the present invention, there is provided amorphous saroglitazar calcium salts.

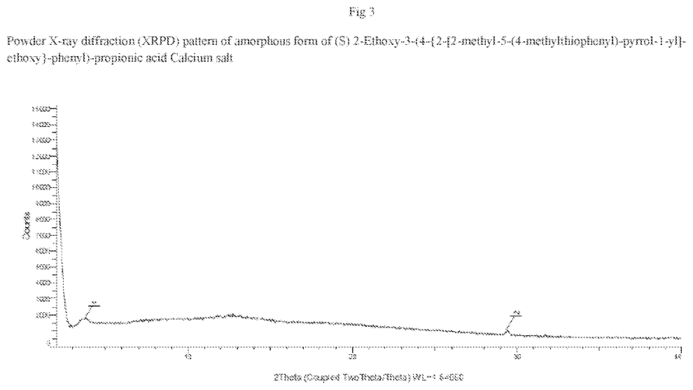

In a further embodiment of the invention is disclosed amorphous form of (S) 2-Ethoxy-3-(4-{2-[2-methyl-5-(4-methylthiophenyl)-pyrrol-1-yl]-ethoxy}-phenyl)-propionic acid calcium salt, which has at least one of the following characteristics:
i) a powder X-ray diffraction pattern substantially in accordance with FIG. 3;
ii) a powder X-ray diffraction pattern having peaks at about 3.740, 29.368±0.2 degrees 2-theta.

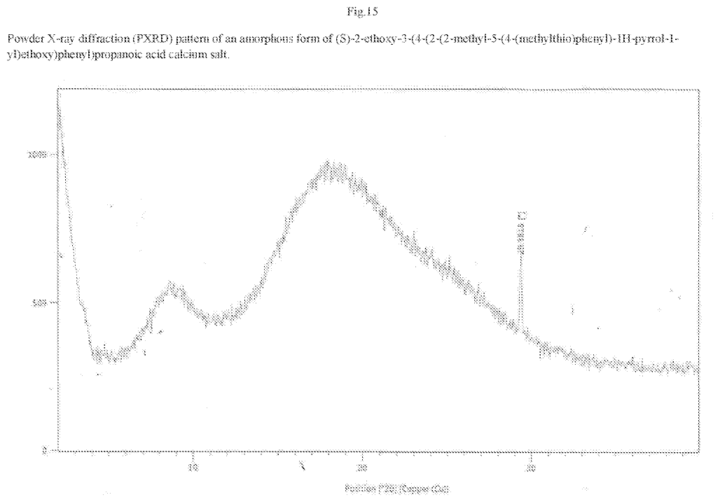

In an embodiment of the present invention is provided a amorphous form of (S)-2-ethoxy-3-(4-acid calcium salt which has at least one of the following characteristics:
i) An amorphous form of (S)-2-ethoxy-3-(4-(2-(2-methyl-5-(4-(methylthio)phenyl)-1H-pyrrol-1-yl)ethoxy)phenyl)propanoic acid calcium salt has a PXRD pattern as per FIG. 15;
ii) A powder XRD pattern having peaks at 29.381±0.2 degrees 2-theta;
iii) An amorphous form of (S)-2-ethoxy-3-(4-(2-(2-methyl-5-(4-(methylthio)phenyl)-1H-pyrrol-1-yl)ethoxy)phenyl)propanoic acid calcium salt has a melting point of 214.6° C.

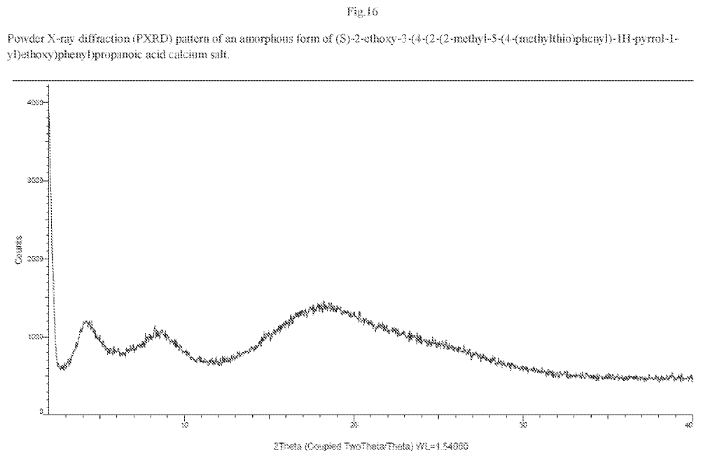

In an embodiment of the present invention is provided a novel amorphous form of (S)-2-ethoxy-3-(4-(2-(2-methyl-5-(4-(methylthio)phenyl)-1H-pyrrol-1-yl)ethoxy)phenyl)propanoic acid calcium salt, which has at least one of the following characteristics:
i) The amorphous form of (S)-2-ethoxy-3-(4-(2-(2-methyl-5-(4-(methylthio)phenyl)-1H-pyrrol-1-yl)ethoxy)phenyl)propanoic acid calcium salt has PXRD pattern as per FIG. 16;
ii) The amorphous form of (S)-2-ethoxy-3-(4-(2-(2-methyl-5-(4-(methylthio)phenyl)-1H-pyrrol-1-yl)ethoxy)phenyl)propanoic acid calcium salt has melting point 175.2° C.

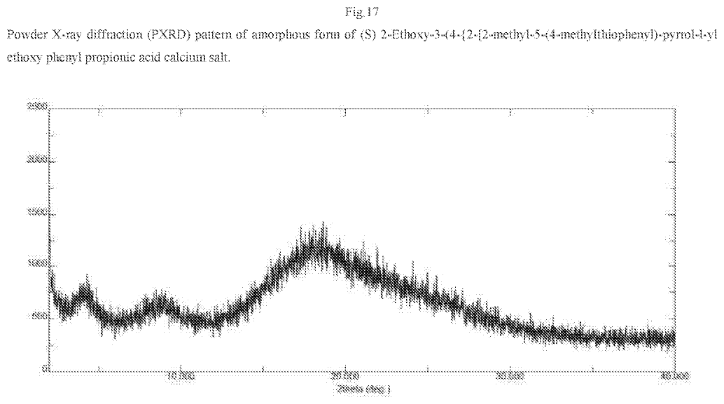

In an embodiment of the present invention is provided a novel amorphous form (S) 2-Ethoxy-3-(4-{2-[2-methyl-5-(4-methylthiophenyl)-pyrrol-1-yl ethoxy phenyl propionic acid calcium which has at least one of the following characteristics:
i) The amorphous form of (S) 2-Ethoxy-3-(4-{2-[2-methyl-5-(4-methylthiophenyl)-pyrrol-1-yl ethoxy phenyl propionic acid calcium salt has PXRD pattern as per FIG. 17;
ii) The amorphous form (S) 2-Ethoxy-3-(4-{2-[2-methyl-5-(4-methylthiophenyl)-pyrrol-1-yl ethoxy phenyl propionic acid calcium salt has melting point 150.6° C.

In another set of the embodiments, the present invention provides certain new salts of compound (I) also represented by the formula (Ia).

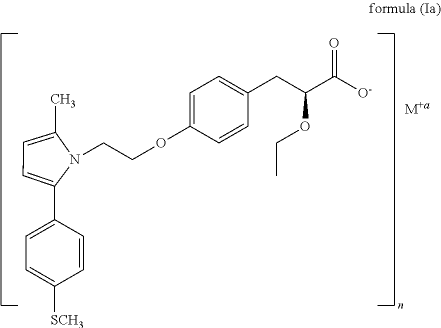

wherein M^{+a} is a cation selected from Cesium (Cs), Copper (Cu), Cobalt (Co), Iron (Fe), Manganese Mn) and Lead (Pb), Aluminum (Al), metformin or metformin-1-glutamic acid ions and n is an integer selected from 1, 2, 3.

In an embodiment these salts may be present either in crystalline or amorphous form or suitable mixtures of crystalline and amorphous forms. In a further embodiment, each of the crystalline and/or amorphous forms may independently exist either in hydrated, solvated, non-solvated, anhydrous, solvent free or desolvated solvates of either the crystalline, amorphous or various mixtures of crystalline and amorphous forms.

In one embodiment some of the novel salts of the present invention can be used for the purification of free acid of formula (I) by reacting the impure acid with suitable salt in a suitable solvent and then the pure acid is obtained from the salt by suitable techniques. The pure free acid of Formula (I) can be further converted to the salts of compound of formula (Ia).

In an embodiment is provided(S) 2-Ethoxy-3-(4-{2-[2-methyl-5-(4-methylthiophenyl)-pyrrol-1-yl]ethoxy}-phenyl)-propionic acid Cesium (Cs) salt. In an embodiment, (S) 2-Ethoxy-3-(4-{2-[2-methyl-5-(4-methylthiophenyl)-pyrrol-1-yl]-ethoxy}-phenyl)-propionic acid Cesium (Cs) salt can be present in either crystalline and/or amorphous form each of which can optionally be present in anhydrous, hydrated or solvated forms.

In an embodiment is provided (S) 2-Ethoxy-3-(4-{2-[2-methyl-5-(4-methylthiophenyl)-pyrrol-1-yl]-ethoxy}-phenyl)-propionic acid Copper (Cu) salt. In an embodiment, (S) 2-Ethoxy-3-(4-{2-[2-methyl-5-(4-methylthiophenyl)-pyrrol-1-yl]-ethoxy}-phenyl)-propionic acid Copper (Cu) salt can be present in either crystalline and/or amorphous form each of which can optionally be present in anhydrous, hydrated or solvated forms.

In an embodiment is provided (S) 2-Ethoxy-3-(4-{2-[2-methyl-5-(4-methylthiophenyl)-pyrrol-1-yl]-ethoxy}-phenyl)-propionic acid Cobalt (Co) salt. In a further embodiment, (S) 2-Ethoxy-3-(4-{2-[2-methyl-5-(4-methylthiophenyl)-pyrrol-1-yl]-ethoxy}-phenyl)-propionic acid Cobalt (Co) salt can be present in either crystalline and/or amorphous form each of which can optionally be present in anhydrous, hydrated or solvated forms.

In one embodiment is provided (S) 2-Ethoxy-3-(4-{2-[2-methyl-5-(4-methylthiophenyl)-pyrrol-1-yl]ethoxy}-phenyl)-propionic acid Iron (Fe) salt. In an embodiment, (S) 2-Ethoxy-3-(4-{2-[2-methyl-5-(4-methylthiophenyl)-pyrrol-1-yl]-ethoxy}-phenyl)-propionic acid Iron (Fe) salt can be present in either crystalline and/or amorphous form each of which can optionally be present in anhydrous, hydrated or solvated forms.

In an embodiment is provided (S) 2-Ethoxy-3-(4-{2-[2-methyl-5-(4-methylthiophenyl)-pyrrol-1-yl]-ethoxy}-phenyl)-propionic acid Manganese (Mn) salt. In a further embodiment, (S) 2-Ethoxy-3-(4-{2-[2-methyl-5-(4-methylthiophenyl)-pyrrol-1-yl]-ethoxy}-phenyl)-propionic acid Manganese (Mn) salt can be present in either crystalline and/or amorphous form each of which can optionally be present in anhydrous, hydrated or solvated forms.

In an embodiment is provided (S) 2-Ethoxy-3-(4-{2-[2-methyl-5-(4-methylthiophenyl)-pyrrol-1-yl]-ethoxy}-phenyl)-propionic acid Lead (Pb) salt. In an embodiment, (S) 2-Ethoxy-3-(4-{2-[2-methyl-5-(4-methylthiophenyl)-pyrrol-1-yl]-ethoxy}-phenyl)-propionic acid Lead (Pb) salt can be present in either crystalline and/or amorphous form each of which can optionally be present in anhydrous, hydrated, solvated or non-solvated forms.

In one embodiment of the present invention is provided Metformin-1-glutamic acid-(S)-2-ethoxy-3-(4-(2-(2-methyl-5-(4-(methylthio)phenyl)-1H-pyrrol-1-yl)ethoxy)phenyl)propanoic acid salt.

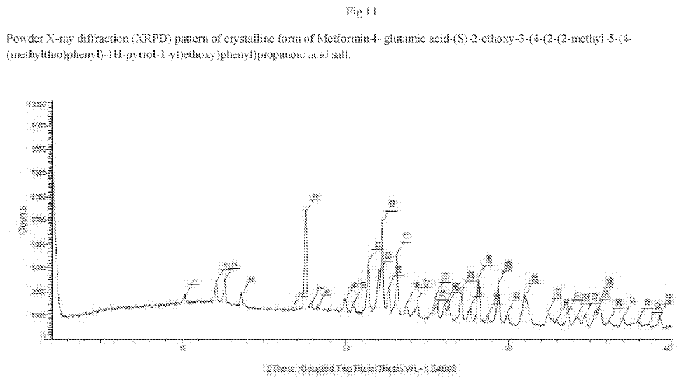

In a further embodiment of the invention disclosed Metformin-1-glutamic acid-(S)-2-ethoxy-3-(4-(2-(2-methyl-5-(4-(methylthio)phenyl)-1H-pyrrol-1-yl)ethoxy)phenyl)propanoic acid salt which has at least one of the following characteristics:
i) a powder X-ray diffraction pattern substantially in accordance with FIG. 11;
ii) a powder X-ray diffraction pattern having peaks at about 17.518, 21.370, 22.198, 23.108±0.2 degrees 2-theta;
iii) a powder X-ray diffraction pattern having additional peaks at about 10.181, 12.100, 12.611, 13.658, 19.924, 20.419, 21.981, 22.592, 23.695, 24.373, 25.516, 26.077, 26.276, 27.061, 27.568, 28.146, 28.697, 29.358, 29.912, 30.971, 32.413, 32.969, 33.645, 34.208, 34.683, 35.355, 35.590, 36.176, 37.031, 37.956, 39.282±0.2 degrees 2-theta.

The crystalline form of Metformin-1-glutamic acid-(S)-2-ethoxy-3-(4-(2-(2-methyl-5-(4-(methylthio)phenyl)-1H-pyrrol-1-yl)ethoxy)phenyl)propanoic acid salt characterized by a melting point 99.2° C.±2° C.

In an embodiment of the present invention is provided an (S)-2-ethoxy-3-(4-(2-(2-methyl-5-(methylthio)phenyl)-1H-pyrrol-1-yl)ethoxy)phenyl)propanoic acid aluminum salt.

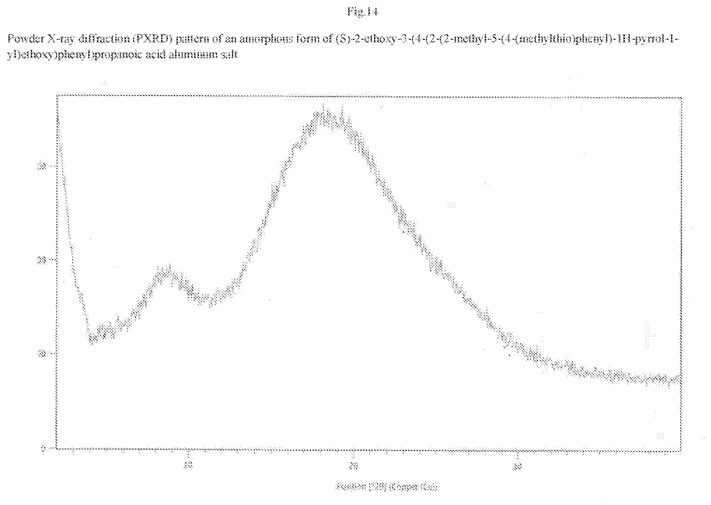

In another embodiment of the present invention is provided an (S)-2-ethoxy-3-(4-(2-(2-methyl-5-(4-(methylthio)phenyl)-1H-pyrrol-1-yl)ethoxy)phenyl)propanoic acid aluminum salt which has at least one of the following characteristic:
i) (S)-2-ethoxy-3-(4-(2-(2-methyl-5-(4-(methylthio)phenyl)-1H-pyrrol-1-yl)ethoxy)phenyl)propanoic acid aluminum salt has PXRD pattern as per FIG. 14;
ii) (S)-2-ethoxy-3-(4-(2-(2-methyl-5-(4-(methylthio)phenyl)-1H-pyrrol-1-yl)ethoxy)phenyl)propanoic acid aluminum salt has melting point 248.1° C.

### General Process for the Preparation of New Salts

In another embodiment the present invention discloses the process for the preparation of pharmaceutically acceptable salts of compound of formula (Ia).

The process comprising:

(a) Compound of formula (I) is dissolved in appropriate solvent;
(b) Dissolving salts source in water or suitable organic solvents;
(c) mix both the solution and stirred at heating or room temperature to get clear solution;
(d) Cool the solution of step (c) to room temperature and filter it or distil it or lyophilize it to get salts of compound of formula (Ia).

Salt source means different counterpart taken to prepare a salt.

The inventors also have developed a process for the preparation of pharmaceutically acceptable saroglitazar salts formula (Ia) using one or more suitable solvents and obtaining a solution of salts source in one or more suitable solvents or water or mixture thereof. The mixing of both the solution may be done by mixing procedure known in the art.

In one aspect, the process may include cooling the solution obtain after step mixing the solution of compound of formula (I) and a solution of salts source in a known manner. The solution of compound of formula (Ia) and a solution of salts source may be obtained by heating the solvent. It may be heated from about 25° C. to reflux temperature.

The term “solvent” includes one or more of alcohols selected from methanol, ethanol, isopropanol, 2-propanol, 1-butanol, and t-butyl alcohol; ethers selected from tetrahydrofuran, 1,4-dioxane, diisopropyl ether, diethyl ether, and methyl tert-butyl ether; water or mixture thereof.

The solvent may be removed by a technique which includes, for example, distillation, distillation under vacuum, lyophilization, evaporation, filtration, filtration under vacuum, decantation and centrifugation.

The product obtained may be further or additionally dried to achieve the desired moisture values. For example, the product may be further or additionally dried in a tray drier, dried under vacuum and/or in a Fluid Bed Drier.

The present invention also provides co-precipitates or premix comprising saroglitazar or pharmaceutically acceptable salts of saroglitazar and a pharmaceutically acceptable excipient.

In one embodiment, the pharmaceutically acceptable excipient is selected from the group consisting of copovidone, polyvinylpyrrolidone (povidone), magnesium aluminometasilicate (neusilin), hydroxypropyl methylcellulose (HPMC), lactose monohydrate and microcrystalline cellulose, polyvinyl alcohol, methyl cellulose, carboxymethyl cellulose, sodium carboxymethyl cellulose, eutragit, soluplus, hydroxyethylcellulose, polyvinyl acetate, maltodextrins, cyclodextrine, gelatins, hydroxypropyl methylcellulose acetate succinate, sugars, zeolite, calcium phosphate, tungstic acid, diatomaceous earth and/or combinations thereof.

In one embodiment, present invention provides premix of saroglitazar or pharmaceutically acceptable salt of saroglitazar with secondary pharmaceutical agent. In a preferred embodiment, secondary pharmaceutical agent used for premix is metformin.

In an embodiment, co-precipitates or premix comprising saroglitazar or pharmaceutically acceptable salt of saroglitazar are in amorphous or crystalline form.

According to another aspect, there are provided pharmaceutical compositions comprising the co-precipitates or premix of saroglitazar or pharmaceutically acceptable salt of saroglitazar disclosed herein, and one or more pharmaceutically acceptable excipients.

In an embodiment there is provided amorphous co-precipitates or premix of saroglitazar or pharmaceutically acceptable salt of saroglitazar.

The amorphous co-precipitates or premix of Saroglitazar or pharmaceutically acceptable salt of Saroglitazar obtained by the processes disclosed herein may be characterized by one or more of their powder X-ray diffraction (XRD) pattern.

In one embodiment is disclosed the co-precipitate or premix of saroglitazar or pharmaceutically acceptable salt of Saroglitazar with magnesium aluminometasilicate (Neusilin).

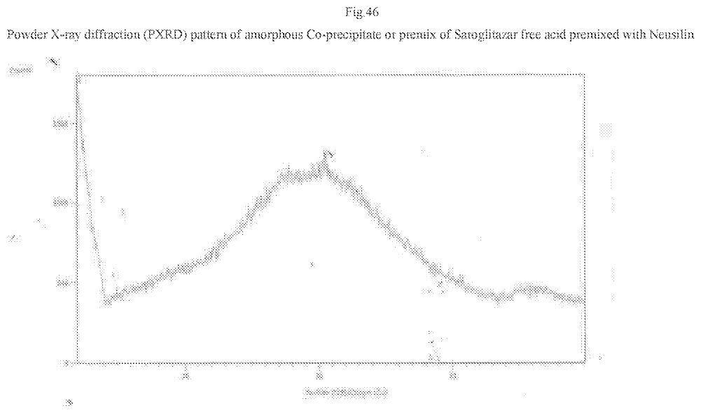

In one embodiment, the amorphous co-precipitate or premix of saroglitazar with magnesium aluminometasilicate (Neusilin) is synthesized and then characterized by a powder XRD and has a pattern substantially in accordance with FIG. 46. The X-ray powder diffraction patterns show a plain halo with no well-defined peaks, thus demonstrating the amorphous nature of the product. In another embodiment, the ratio of saroglitazar acid or salt to amount of magnesium aluminometasilicate Neusilin) is selected from the range of 1:0.1-1:5, specifically 1:0.6 and 1:1.

In one embodiment is disclosed the co-precipitate or premix of Saroglitazar or pharmaceutically acceptable salt of Saroglitazar is with copovidone.

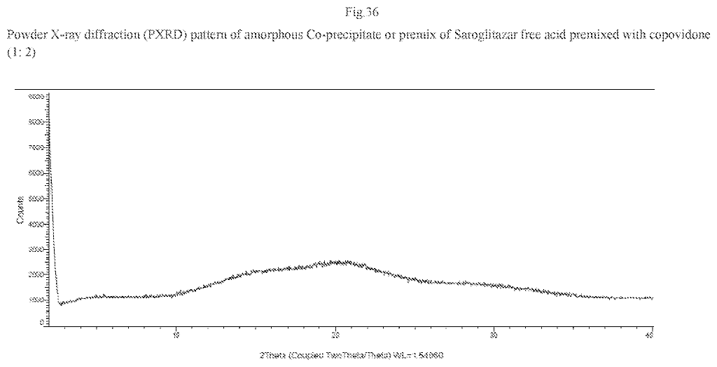

In one embodiment, the amorphous co-precipitate or premix of Saroglitazar with copovidone is synthesized and further characterized by a powder XRD and has a pattern substantially in accordance with FIG. 36. The X-ray powder diffraction patterns show a plain halo with no well-defined peaks, thus demonstrating the amorphous nature of the product. In another embodiment, ratio of saroglitazar acid or salt to amount of copovidone is selected from the range of 1:0.1-1:5, specifically 1:1.2, 1:1, 1:2, 1:3 and 1:5.

In one embodiment is disclosed the co-precipitate or premix of saroglitazar or pharmaceutically acceptable salt of Saroglitazar is with HPMC.

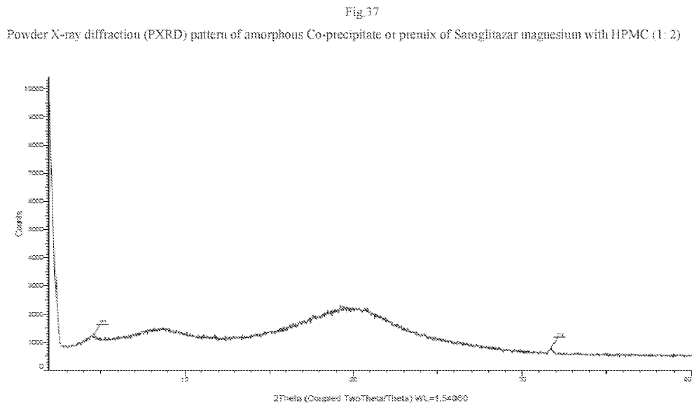

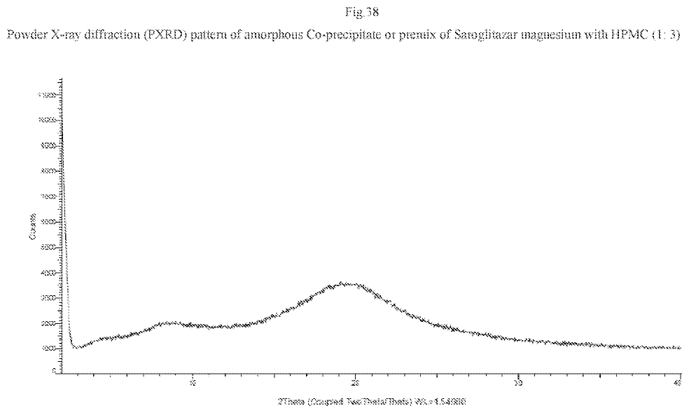

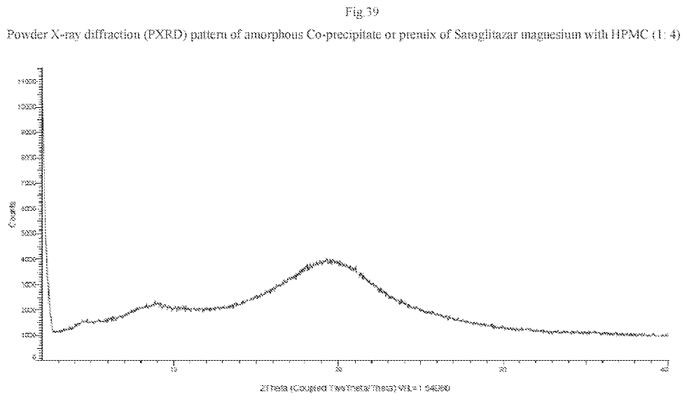

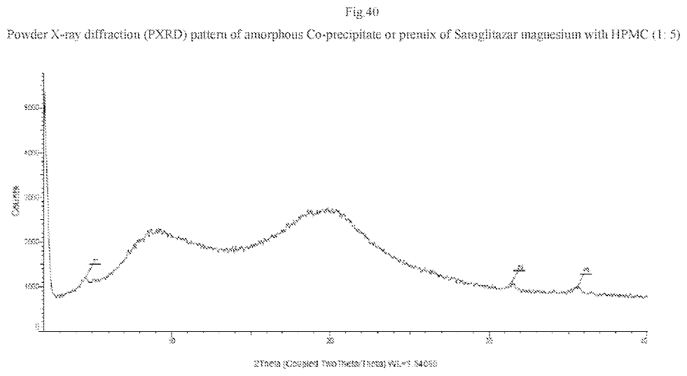

In one embodiment, the various amorphous co-precipitate or premix of saroglitazar or saroglitazar magnesium with HPMC is synthesized and characterized by a powder XRD. The XRD pattern obtained for the various co-precipitates or premixes show patterns substantially in accordance with FIGS. 37, 38, 39 and 40 respectively. The X-ray powder diffraction patterns of each of the co-precipitates or premixes shows plain halo with no well-defined peaks, thus demonstrating the amorphous nature of the products. In another embodiment, ratio of saroglitazar acid or salt to amount of HPMC is selected from the range of 1:0.1-1:5, specifically 1:2, 1:3, 1:4 and 1:5.

In one embodiment is disclosed the co-precipitate or premix of saroglitazar or pharmaceutically acceptable salt of saroglitazar with HPMC.AS.

In one embodiment, the co-precipitate or premix of saroglitazar with HPMC.AS is synthesized. In another embodiment, ratio of saroglitazar to amount of HPMC. AS is selected from the range of 1:0.1-1:5, specifically 1:1.2, 1:2, 1:3 and 1:5.

In one embodiment is disclosed, the co-precipitate or premix of Saroglitazar or pharmaceutically acceptable salt of saroglitazar with methacrylic acid.

In an embodiment, the novel co-precipitate or premixes of saroglitazar acid premixed with methacrylic acid (Eudragit) are synthesized. In another embodiment, ratio of saroglitazar to amount of methacrylic acid (Eudragit) is selected from the range of 1:0.1-1:5, specifically 1:2, 1:3, 1:4 and 1:5.

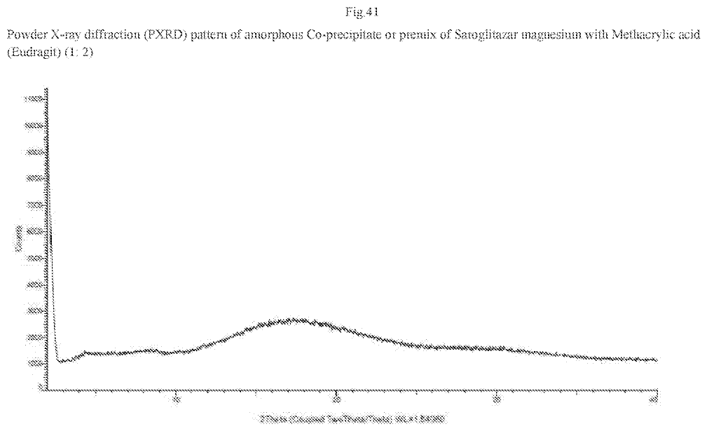

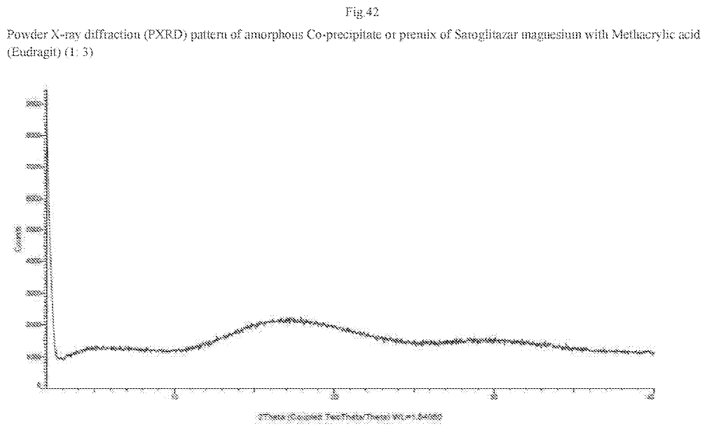

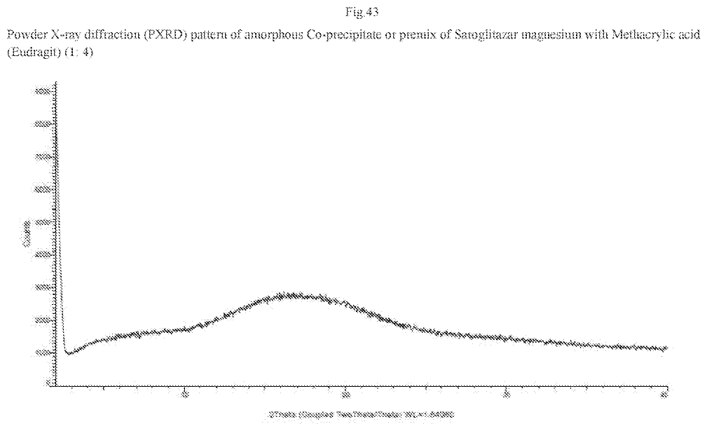

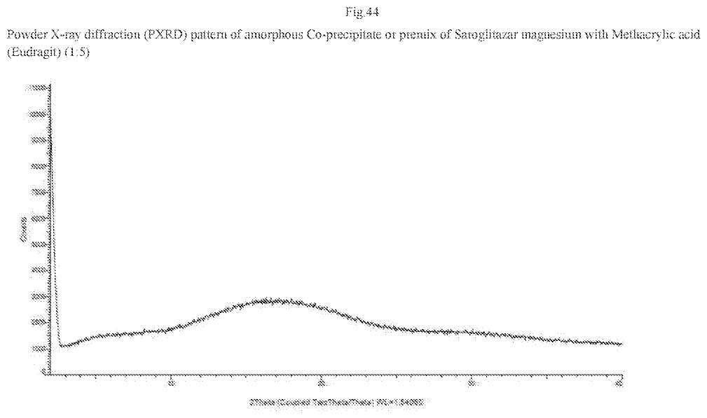

In an embodiment is provided the novel amorphous co-precipitate or premix of saroglitazar magnesium premixed with methacrylic acid (Eudragit) are synthesized and characterized by a powder XRD pattern. The XRD pattern obtained for the various co-precipitates or premixes are substantially in accordance with FIGS. 41, 42, 43, 44 respectively. The powder X-ray diffraction patterns show a plain halo with no well-defined peaks, thus demonstrating the amorphous nature of the products. In another embodiment, ratio of saroglitazar magnesium to amount of methacrylic acid (Eudragit) is selected from the range of 1:0.1-1:5, specifically 1:2, 1:3, 1:4 and 1:5.

In one embodiment is disclosed the co-precipitate or premix of Saroglitazar or pharmaceutically acceptable salt of saroglitazar with metformin.

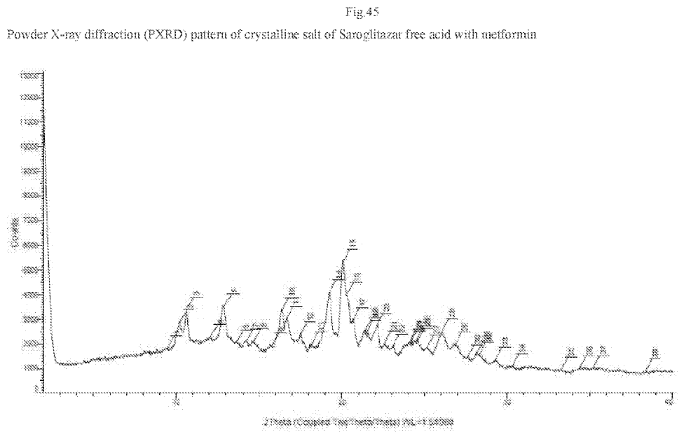

In an embodiment, there is provided the novel co-precipitate or premix of saroglitazar acid with metformin salt. In another embodiment the ratio of saroglitazar acid to amount of metformin salt is 1:1. The XRD pattern obtained for the premix is substantially in accordance with FIG. 45.

In one embodiment is disclosed the co-precipitate or premix of Saroglitazar or pharmaceutically acceptable salt of Saroglitazar with diatomaceous earth.

In an embodiment, there is provided a premix of saroglitazar and diatomaceous earth. In another embodiment, the ratio of saroglitazar acid to diatomaceous earth is 1:4.

In one embodiment, the co-precipitate or premix of saroglitazar or pharmaceutically acceptable salt of saroglitazar with zeolite.

In an embodiment, there is provided a novel premix of saroglitazar and zeolite. In another embodiment, ratio of saroglitazar acid to zeolite is 1:3.

In one embodiment, the co-precipitate or premix of Saroglitazar or pharmaceutically acceptable salt of saroglitazar with tribasic calcium phosphate.

In an embodiment, there is provided a novel premix of saroglitazar and tribasic calcium phosphate. In another embodiment, ratio of saroglitazar to tribasic calcium phosphate is 1:4.

In one embodiment, the co-precipitate or premix of Saroglitazar or pharmaceutically acceptable salt of saroglitazar with tungstic acid.

In an embodiment, there is provided a novel premix of saroglitazar and tungstic acid. In another embodiment, ratio of saroglitazar acid to tungstic acid is 1:4.

According to another aspect, there is provided a process for preparing an co-precipitate or premix of Saroglitazar or pharmaceutically acceptable salt of saroglitazar and a pharmaceutically acceptable excipient, comprising:

a) providing a solution of Saroglitazar free acid or pharmaceutically acceptable salt of Saroglitazar and a pharmaceutically acceptable excipient in a solvent wherein the solvent is water, an organic solvent, or a solvent medium comprising water and an organic solvent; wherein the organic solvent is selected from the group consisting of an alcohol, a ketone, a halogenated hydrocarbon, a nitrile, an ester, an organic water-miscible solvent, and mixtures thereof;
b) Optionally, filtering the solvent solution to remove insoluble matter; and
c) Substantially removing the solvent from the solution to produce the amorphous co-precipitate or premix of Saroglitazar free acid or pharmaceutically acceptable salt of Saroglitazar with the pharmaceutically acceptable excipient.

The novel salts, premixes and novel forms of known salts of the present invention can be formulated into suitable pharmaceutically acceptable compositions by combining with suitable excipients by techniques and processes and concentrations as are well known.

The quantity of active component, that is, novel salts, premixes and novel forms of known salts according to this invention, in the pharmaceutical composition and unit dosage form thereof may be varied or adjusted widely depending upon several factors such as the particular application method, the potency of the particular compound and the desired concentration

The novel salts, premixes and novel forms of known salts of the present invention can be formulated into suitable pharmaceutically acceptable compositions by combining with suitable excipients by techniques and processes and concentrations as are well known.

The pharmaceutical compositions according to this invention can exist in various forms. In some embodiments, the pharmaceutical composition is in the form of a powder or solution. In some other embodiments, the pharmaceutical compositions according to the invention are in the form of a powder that can be reconstituted by addition of a compatible reconstitution diluent prior to parenteral administration. Non-limiting example of such a compatible reconstitution diluents include water.

The pharmaceutical compositions are prepared and formulated according to conventional methods, such as those disclosed in standard reference texts and are well within the scope of a skilled person. For example, the solid oral compositions may be prepared by conventional methods of blending, filling or tableting. Repeated blending operations may be used to distribute the active agent throughout those compositions employing variable quantities of fillers, binding agent, lubricants, glidants, disintegrants, etc. Such operations are of course conventional in the art. The tablets may be coated according to methods well known in normal pharmaceutical practice.

Examples of binding agents include acacia, alginic acid, carboxymethylcellulose calcium, carboxymethylcellulose sodium, dextrates, dextrin, dextrose, ethylcellulose, gelatin, liquid glucose, guar gum, hydroxyethyl cellulose, hydroxypropyl cellulose, hydroxypropyl methylcellulose, magnesium aluminium silicate, maltodextrin, methyl cellulose, polymethacrylates, polyvinylpyrrolidone, pregelatinised starch, sodium alginate, sorbitol, starch, syrup, tragacanth.

Examples of fillers include calcium carbonate, calcium phosphate, calcium sulphate, carboxymethylcellulose calcium, carboxymethylcellulose sodium, compressible sugar, confectioner's sugar, dextrates, dextrin, dextrose, dibasic calcium phosphate dihydrate, dibasic calcium phosphate, fructose, glyceryl palmitostearate, glycine, hydrogenated vegetable oil-type 1, kaolin, lactose, maize starch, magnesium carbonate, magnesium oxide, maltodextrin, mannitol, microcrystalline cellulose, polymethacrylates, potassium chloride, powdered cellulose, pregelatinised starch, sodium chloride, sorbitol, starch, sucrose, sugar spheres, talc, tribasic calcium phosphate, xylitol.

Examples of lubricants include calcium stearate, glyceryl monostearate, glyceryl palmitostearate, magnesium stearate, microcrystalline cellulose, sodium benzoate, sodium chloride, sodium lauryl sulphate, stearic acid, sodium stearyl fumarate, talc, zinc stearate.

Examples of glidants include colloidal silicon dioxide, powdered cellulose, magnesium trisilicate, silicon dioxide, talc.

Examples of disintegrants include alginic acid, carboxymethylcellulose calcium, carboxymethylcellulose sodium, colloidal silicon dioxide, croscartmellose sodium, crospovidone, guar gum, magnesium aluminium silicate, microcrystalline cellulose, methyl cellulose, polyvinylpyrrolidone, polacrilin potassium, Pregelatinized starch, sodium alginate, sodium lauryl sulphate, sodium starch glycollate.

Use of different amorphous, partially crystalline and crystalline salts of Saroglitazar sodium and Saroglitazar magnesium, different amorphous form of pharmaceutically acceptable salts of saroglitazar, wherein positive ion is selected from lithium, potassium and calcium, certain novel salts of Saroglitazar formula (I), new co-precipitates or premix form of Saroglitazar or pharmaceutically acceptable salt of Saroglitazar for the treatment of dyslipidemia or hyperglycemia.

Following characterization methods were used to confirm synthesis of novel salts, premixes and novel forms of known salts.

### Analytical Methods

### i) Chromatographic Purity/Related Substance (by HPLC)

Column: YMC-pack ODS-AM (type L₁) or equivalent; 250×4.6 mm, 5 μm
Wave length: 294 nm
Column temp.: 30° C.
Mobile phase: [Ammonium acetate buffer: Acetonitrile]/[55:45]
Flow rate: 1 ml/min
Injection vol.: 5 μl
Retention time: About 10 mints.
Run time: 60 mints.

### ii) Chromatographic Purity Chiral (by HPLC)

Column: chiralcel OJ-H: 250×4.6 mm, 5 μm
Wave length: 294 nm
Column temp.: 35° C.
Mobile phase: 0.05% TFA in EtOH:Hexane/(12:88)
Flow rate: 0.8 ml/min
Injection vol.: 5 μl
Run time: 60 mints.
   iii) XRPD Method

Sample Preparation: Place a Sufficient quantity of sample to be analyzed on the sample holder plate and flatten it with the help of another plate to achieve a smooth surface. Record the diffraction pattern as per below instrumental parameters

| Instrument used | 2k W XRD |
|---|---|
| Model | MF2100 |
| Make | Rigaku |
| Instrument Parameter | |
| 1. X-ray | Cu/40 kV/30 mA |
| 2. Diversion slit | 1° |
| 3. Scattering slit | 1° |
| 4. Receiving slit | 0.15 mm |
| 5. Filter | Ni-kβ filter |
| 6. Counter | Scintillation counter |
| 7. Scan mode | Continuous |
| 8. Scan speed | 4.000°/minute |
| 9. Sampling width | 0.010° |
| 10. Scan axis | 2theta\theta |
| 11. Scan range | 2.0° to 40.0° |
| 12. Theta offcet | 0.000° |

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of a person skilled in the art.

### EXAMPLES

### Example—1

### Preparation of (S)-2-ethoxy-3-(4-(2-(2-methyl-5-(4-(methylthio)phenyl)-1H-pyrrol-1-yl)ethoxy)phenyl)propanoic Acid Lithium Salt

To a solution of (S)-2-ethoxy-3-(4-(2-(2-methyl-5-(4-(methylthio)phenyl)-1H-pyrrol-1-yl)ethoxy)phenyl)propanoic acid (1.0 g, 2.275 mmol) in MeOH (20 ml), lithium hydroxide hydrate (0.095 g, 2.275 mmol) was added and the reaction mixture was stirred at 30° C. for 20 hrs. Then the reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid lithium salt as a white solid. Yield: 993 mg (98.0% yield), HPLC Purity: 97.20% Melting point: 131.2° C.

### Example—2

### Preparation of (S)-2-ethoxy-3-(4-(2-(2-methyl-5-(4-(methylthio)phenyl)-1H-pyrrol-1-yl)ethoxy)phenyl)propanoic Acid Potassium Salt

To a solution of (S)-2-ethoxy-3-(4-(2-(2-methyl-5-(4-(methylthio)phenyl)-1H-pyrrol-1-yl)ethoxy)phenyl)propanoic acid (925 mg, 2.104 mmol) in MeOH (20 ml), KOH (118 mg, 2.104 mmol) was added and the reaction mixture was stirred at 30° C. for 5.0 hrs. Then the reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid potassium salt as a yellow solid. Yield: 975 mg (97.0% yield), HPLC Purity: 93.42 Melting point: 62.1° C.

### Example—3

### Preparation of (S)-2-ethoxy-3-(4-(2-(2-methyl-5-(4-(methylthio)phenyl)-1H-pyrrol-1-yl)ethoxy)phenyl)propanoic Acid Calcium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (3.2 g, 6.93 mmol) in MeOH (45 ml), Calcium acetate (1.426 g, 9.01 mmol) was added with followed by addition of 100 ml water and the reaction mixture was stirred at 30° C. for 1.0 hr. Solid separated was filtered through Buchner funnel, washed with water and dried over P₂O₅ under vacuum to yield as white solid. The solid was dissolved in dichloromethane and evaporated under vacuum and dried to yield (S)-2-ethoxy-3-(4-(2-(2-methyl-5-(4-(methylthio)phenyl)-1H-pyrrol-1-yl)ethoxy)phenyl)propanoic acid calcium salt as yellow solid. Yield: 3.45 g (96.4%), HPLC: purity: 97.93%, melting point: 197.8° C.

### Example—4

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (2.0 g, 4.55 mmol) in MeOH (40 ml), sodium hydroxide (0.182 g, 4.55 mmol) was added and the reaction mixture was stirred at 30° C. for 24 hrs. The reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid sodium salt as a yellow solid. Yield: 2.069 g (98.5% yield), HPLC Purity: 97.38%, Melting point: 238.3° C.

### Example—5

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (1.0 g, 2.275 mmol) in ethanol (30 ml), sodium hydroxide (0.091 g, 2.275 mmol) was added and the reaction mixture was stirred at 30° C. for 24 hrs. The reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid sodium salt as yellow solid. Yield: 995 mg (94.8% yield), HPLC Purity: 96.83%, Melting point: 263.2° C.

### Example—6

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (1.1 g, 2.502 mmol) in n-Butanol (50 ml), sodium hydroxide (0.100 g, 2.502 mmol) was added and the reaction mixture was stirred at 30° C. for 24 hrs. The reaction mixture was concentrated under vacuums to yield (S)-2-ethoxy-3-(4-(2-(2-methyl-5-(4-(methylthio)phenyl)-1H-pyrrol-1-yl)ethoxy)phenyl)propanoic acid sodium salt as yellow solid. Yield: 1.100 g (95% yield), HPLC Purity: 97.39%, Melting point: 240.6° C.

### Example—7

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of sodium salt of (S)-2-ethoxy-3-(4-(2-(2-methyl-5-(4-(methylthio)phenyl)-1H-pyrrol-1-yl)ethoxy)phenyl)propanoic acid (250 mg, 0.561 mmol) in 5% NaCl (5 ml), magnesium sulfate (150 mg) in water (2.0 ml) was added and the reaction mixture was stirred at 30° C. for 48 hrs. Water from the reaction mixture was decanted and the residue was washed with water. Then residue was dissolved in 10% methanol in chloroform. Then organic layer was concentrated and dried under vacuum to yield (S)-2-ethoxy-3-(4-(2-(2-methyl-5-(4-(methylthio)phenyl)-1H-pyrrol-1-yl)ethoxy)phenyl)propanoic acid magnesium salt as a yellow solid.

Yield: 266 mg (97.2% yield), HPLC Purity: 95.79%, Melting point: 148.4° C.

### Example—8

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of sodium salt of (S)-2-ethoxy-3-(4-(2-(2-methyl-5-(4-(methylthio)phenyl)-1H-pyrrol-1-yl)ethoxy)phenyl)propanoic acid (250 mg, 0.561 mmol) in MeOH (10 ml) into the reaction flask. Then in this magnesium sulfate (150 mg) in water (2.0 ml) was added and the reaction mixture was stirred at 30° C. for 20 hours. The reaction mixture was filtered and the filtrate was concentrated under reduced pressure then residue was stirred in acetone (20 ml) and stirred for 2.0 hrs at room temp then the solid was filtered and dried over P₂O₅ to yield (S)-2-ethoxy-3-(4-(2-(2-methyl-5-(4-(methylthio)phenyl)-1H-pyrrol-1-yl)ethoxy)phenyl)propanoic acid Magnesium salt as a yellow solid.

Yield: 210 mg (77.0% yield), HPLC Purity: 96.0%, Melting point: 223-224° C.

### Example—9

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

In a dry, 250 mL round bottom flask 80 ml methanol was taken. To this 20 g (S)-2-ethoxy-3-(4-(2-(2-methyl-5-(4-(methylthio)phenyl)-1H-pyrrol-1-yl)ethoxy)phenyl)propanoic acid ethyl ester was added at room temperature, under nitrogen. To this 1.89 g sodium hydroxide dissolved in 20 mL water was added and stirred at room temperature for 3 hours to complete hydrolysis. Solvent was removed under reduced pressure. 150 ml water was added to the material. Impurity was removed by solvent washing. To aqueous layer was added 5 g magnesium acetate tetra hydrate (dissolved in 20 ml water) and stirred with for 15 min. Sticky material was extracted with dichloromethane and subsequently add n-heptane to precipitate (S)-2-ethoxy-3-(4-(2-(2-methyl-5-(4-(methylthio)phenyl)-1H-pyrrol-1-yl)ethoxy)phenyl)propanoic acid magnesium salt. Solid was filtered, and dried to yield 10.3 gm (53%) solid product. Then product (1.0 gm) was dissolved product in MeOH (15 ml): CHCl3 (30.0 ml). Then reaction mixture was concentrated under vacuum and dried to yield (S)-a-ethoxy-4-[2-[-methyl-5-[4-(methylthio)phenyl]-1H-pyrrol-]-yl]ethoxy]benzenepropanoic acid magnesium salt as off white solid. Yield: 990 mg (99.0% yields), Melting point: 185-190° C.

### Example 10

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

In 500 ml round bottom flask. (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (10.0 g, 0.017 mole) was charged. A solution of Ethyl acetate (100 ml) and water (100 ml) was added at RT under N₂ atm., 50% dil HCl (20 ml) solution was added and stirred for 10 minutes. The organic layer was separated and washed with water. The solvent was distilled out using rotavapor under vacuum at 50-55° C. Free acid compound was obtained. In a 250 ml one necked flask, above oily mass (free acid compd.) charged and dissolved in Methanol (15 ml), added Sodium methoxide (0.915 g, 0.016 mole) under nitrogen atmosphere and stirred for 30 minutes at RT. Added drop wise solution of Magnesium acetate tetrahydrate (2.48 g, 0.011 mole) in Methanol (5 ml) under nitrogen atmosphere and stirred for 1 hour at RT. The solvent was distilled out using rotavapor, it was stripped off using DCM (2×30 ml) and removed traces under high vacuum. Solid material was obtained. It was dissolved in DCM (30 ml) and portion wise dumped into n-Heptane (100 ml) under vigorous stirring and stirred at RT. Solvent was decanted and added n-Heptane (100 ml) and stirred at RT for overnight (18 hr). Solid was filtered and washed with n-Heptane and dried the solid using rotavapor under vacuum. Yield: 8.8 g. Light brown colored powder, HPLC: Chemical purity: 97.83%, amorphous form. DSC: 56.86° C., 90.44° C., 219.14° C. TGA: Delta Y=7.8595%

### Example—11

### Preparation of Metformin-1-glutamic acid-(S)-2-ethoxy-3-(4-(2-(2-methyl-5-(4-(methylthio)phenyl)-1H-pyrrol-1-yl)ethoxy)phenyl)propanoic Acid Salt

To a solution of Metformin (0.294 g, 2.275 mmol) in MeOH (5 ml), L-glutamic acid (0.167 g, 1.137 mmol) was added and the reaction mixture was stirred at 30° C. for 30 mins. Then in this solution of (S)-2-ethoxy-3-(4-(2-(2-methyl-5-(4-(methylthio)phenyl)-1H-pyrrol-1-yl)ethoxy)phenyl)propanoic acid (0.500 g, 1.137 mmol) in MeOH (5 ml) was added and the reaction mixture was stirred at 30° C. for 48 hrs. Reaction mixture was concentrated under vacuum to yield solid which was titruted with diisopropyl ether, filtered and dried to yield Metformin-1-glutamic acid-(S)-2-ethoxy-3-(4-(2-(2-methyl-5-(4-(methylthio)phenyl)-1H-pyrrol-1-yl)ethoxy)phenyl)propanoic acid salt as an off white solid.

Yield: 917 g (95.4% yields), Melting point: 99.2° C.

¹HNMR (DMSO-d6) δ1.00 (bs, 3H), 1.85 (bs, 1H), 2.31 (bs, 6H), 2.68-2.85 (m, 3H), 2.93 (bs, 12H) , 3.17-3.27 (m, 2H), 3.40-3.59 (m, 2H), 3.73 (s, 2H), 3.87 (s, 2H), 3.96 (s, 2H), 4.24 (s, 2H), 5.85 (s, 1H), 5.97 (s, 1H), 6.64 (d, J=6.4 Hz, 2H), 6.89 (8H, exchangeable), 7.06 (d, J=6.8 Hz, 2H), 7.17 (4H, exchangeable), 7.33 (m, 4H), 7.61 (2H, exchangeable).

### Example 12

### Preparation of (S)-2-ethoxy-3-(4-(2-(2-methyl-5-(4-(methylthio)phenyl)-1H-pyrrol-1-yl)ethoxy)phenyl)propanoic Acid Lithium Salt

To a solution of (S)-2-ethoxy-3-(4-(2-(2-methyl-5-(4-(methylthio)phenyl)-1H-pyrrol-1-yl)ethoxy)phenyl)propanoic acid (4.9 g, 11.15 mmol) in methanol (50 ml), lithium hydroxide hydrate (0.468 g, 11.15 mmol) was added and the reaction mixture was stirred at 30° C. for 20 hrs. Then the reaction mixture was concentrated under vacuum to yield (S)-2-ethoxy-3-(4-(2-methyl-5-(4-(methylthio)phenyl)-1H-pyrrol-1-yl)ethoxy)phenyl)propanoic acid lithium salt as a white solid. Yield: 5.03 gm (98.0% yield). HPLC Purity: 96.82%, Melting point: 285.5° C.

### Example 13

### Preparation of Amorphous Form (S) 2-Ethoxy-3-(4-{2-[2-methyl-5-(4-methylthiophenyl)-pyrrol-1-yl Ethoxy Phenyl Propionic Acid Potassium Salt

In 250 ml round bottom flask, (S,S)(-,-)α-Methyl benyzlamine salt of 2-ethoxy-3-(4-{2-[2-methyl-5-(4-methylthiophenyl)-pyrrol-1-yl]-ethoxy}-phenyl)-propionic acid (10.0 g, 0.017 mole) was charged. A solution of Ethyl acetate (72 ml) and water (50 ml) was added at RT under N₂ atm., 50% dil HCl (5 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (50 ml), added potassium tert-butoxide (1.81 g, 0.016 mole) under nitrogen atmosphere and stirred for 30 minutes at RT. The solvent was distilled out using rotavapor. Solid material was obtained. Triturated the solid material with Hexane (50 ml) and stirred for 15 minute. Hexane layer was decanted and dissolved the solid again in methanol (40 ml). The solvent was distilled out and dried the solid using rotavapor under vacuum. Yield: 7.6 g, Cream colored powder, HPLC: Chemical purity: 97.29%, Chiral purity: 99.42%, amorphous form.

### Example 14

### Preparation of (S)-2-ethoxy-3-(4-(2-(2-methyl-5-(4-(methylthio)phenyl)-1H-pyrrol-1-yl)ethoxy)phenyl)propanoic Acid Aluminum Salt

To a solution of (S)-2-ethoxy-3-(4-(2-(2-methyl-5-(4-(methylthio)phenyl)-1H-pyrrol-1-acid sodium salt (500 mg, 1.083 mmol) in water (20 ml), Aluminum chloride, anhydrous, granules (50.6 mg, 0.379 mmol) was added and stirred at 30° C. for 30 minute. Then add 30 ml water and the reaction mixture was stirred at 30 for 2.0 hours. Solid separated was filtered through Buchner funnel, washed with water and dried over P₂O₅ under vacuum to yield (S)-2-ethoxy-3-(4-(2-(2-methyl-5-(4-(methylthio)phenyl)-1H-pyrrol-1-yl)ethoxy)phenyl)propanoic acid aluminum salt as a white solid, Yield: 0.375 g (72% yield), HPLC Purity: 96.79%, Melting point: 248.1° C.

### Example 15

### Preparation of (S)-2-ethoxy-3-(4-(2-(2-methyl-5-(4-(methylthio)phenyl)-1H-pyrrol-1-yl)ethoxy)phenyl)propanoic Acid Calcium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (700 mg, 1.57 mmol) in ethanol (42 ml), calcium acetate (312 mg, 1.972 mmol) was added with followed by addition of 12 ml water and the reaction mixture was stirred at 30° C. for 20 hours. Solid was filtered through Buchner funnel, washed with water and dried over P₂O₅ under vacuum to yield as white solid. Then solid was dissolved in dichloromethane and evaporated under vacuum and dried to yield (S)-2-ethoxy-3-(4-(2-(2-methyl-5-(4-(methylthio)phenyl)-1H-pyrrol-1-yl)ethoxy)phenyl)propanoic acid calcium salt as yellow solid. Yield: 400 mg (50.8%), HPLC: purity: 96.29%.

### Example 16

### Preparation of (S)-2-ethoxy-3-(4-(2-(2methyl-5-(4-(methylthio)phenyl)-1H-pyrrol-1-yl)ethoxy)phenyl)propanoic Acid Calcium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (1.0 gm, 2.16 mmol) in methanol (20 ml), calcium chloride (313 mg, 2.82 mmol) was added with followed by addition of 100 ml water and the reaction mixture was stirred at 30° C. for 1.0 hour. Solid separated was filtered through Buchner funnel, washed with water and dried over P₂O₅ under vacuum to yield (S)-2-ethoxy-3-(4-(2-(2-methyl-5-(4-(methylthio)phenyl)-1H-pyrrol-1-yl)ethoxy)phenyl)propanoic acid calcium salt as white solid. Yield: 1.0 gm (93.0%), HPLC purity: 96.83%, melting point: 214.6° C.

### Example 17

### Preparation of (S)-2-ethoxy-3-(4-(2-(2-methyl-5-(4-(methylthio)phenyl)-1H-pyrrol-1-yl)ethoxy)phenyl)propanoic Acid Calcium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (900 mg, 1.950 mmol) in ethanol (40 ml), calcium acetate (401 mg, 2.53 mmol) was added with followed by addition of 60 ml water and the reaction mixture was stirred at 30° C. for 1.0 hours. Solid separated was filtered through Buchner funnel, washed with water and dried over P₂O₅ under vacuum to yield (S)-2-ethoxy-3-(4-(2-(2-methyl-5-(4-(methylthio)phenyl)-1H-pyrrol-1-yl)ethoxy)phenyl)propanoic acid calcium salt as a white solid. Yield: 0.975 g (96.4%), HPLC: purity: 96.16%, Melting point: 175.2° C.

### Example 18

Preparation of amorphous form (S) 2-Ethoxy-3-(4-{2-[2-methyl-5-(4-methylthiophenyl)-pyrrol-1-yl ethoxy phenyl propionic acid Calcium salt

In 250 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (10.0 g, 0.017 mole) was charged. A solution of Ethyl acetate (72 ml) and water (50 ml) was added at RT under N₂ atm., 50% dil HCl (5 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (40 ml), added solution of Sodium hydroxide (0.929 g, 0.023 mole) in Water (40 mL) and stirred for 15 minutes at RT. Added solution of calcium acetate mono hydrate (2.05 g, 0.011 mole) in Water (40 mL) at 10-15° C. and stirred for 30 min at 10-15° C. Precipitated solid was filtered and washed with water. Solid was suck dried under vacuum and then dried over rotavapor under vacuum at 45-50° C. Yield: 8.0 g, Off white colored powder, HPLC: Chemical purity: 98.74%, Chiral purity: 93.19%. M.P.: 150.6° C.

### Example 19

### Preparation of (S)-2-ethoxy-3-4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (1.050 g, 2.39 mmol) in methanol (20 ml), sodium hydroxide (96 mg, 2.39 mmol) was added and the reaction mixture was stirred at 30° C. for 24 hrs. The reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid sodium salt as a yellow solid. Yield: 1.0 g (90.0% yield), HPLC Purity: 91.99%, Melting point: 243.8° C.

### Example 20

### Preparation of (S)-2-ethoxy-3-4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (1.050 g, 2.39 mmol) in methanol (20 ml), solution of sodium hydroxide (96 mg, 2.39 mmol) in water (1.0 ml) was added and the reaction mixture was stirred at 30° C. for 24 hrs. The reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid sodium salt as a yellow solid. Yield: 1.0 g (90.0% yield), HPLC Purity: 91.93%:

### Example 21

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (1.0 g, 2.275 mmol) in tetrahydrofuran (20 ml), sodium (52.3 mg, 2.275 mmol) was added and the reaction mixture was stirred at 30° C. for 72 hours and then heated at 55° C. for 3.0 hours and again stirred at 30° C. for 20 hours. Reaction mixture was concentrated under reduced pressure and the product was titruted with hexane and then dried to yield (S)-2-ethoxy-3-(4-(2-(2-methyl-5-(4-(methylthio)phenyl)-1H-pyrrol-1-yl)ethoxy)phenyl)propanoic acid sodium salt as a yellow solid. Yield: 1.034 g (98.5% yield), HPLC Purity: 95.49%, Melting point: 240.6° C.

### Example 22

### Preparation of (S)-2-ethoxy-3-(4-2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (5.2 g, 11.83 mmol) in methanol (110 ml), sodium hydroxide (0.473 g, 11.83 mmol) was added and the reaction mixture was stirred at 30° C. for 24 hrs. The reaction mixture was concentrated under vacuum to yield (S)-2-ethoxy-3-(4-(2-(2-methyl-5-(4-(methylthio)phenyl)-1H-pyrrol-1-yl)ethoxy)phenyl)propanoic acid sodium salt as a yellow solid. Yield: 5.38 g (98.5% yield), HPLC Purity: 95.31%, Melting point: 139.1° C.

### Example 23

### Preparation of (S)-2-ethoxy-3-(4-(2-(2-methyl-5-(4-(methylthio)phenyl)-1H-pyrrol-4-yl)ethoxy)phenyl)propanoic Acid Sodium Salt

Heat the (S)-2-ethoxy-3-(4-(2-(2-methyl-5-(4-(methylthio)phenyl)-1H-pyrrol-1-yl)ethoxy)phenyl)propanoic acid sodium salt (750 mg, 1.625 mmol) at 100° C. for 6.0 hours. The product was cooled to 30° C. and put overnight at 30° C. Then residue was titruted with diisopropyl ether and dried to yield (S)-2-ethoxy-3-(4-(2-(2-methyl-5-(4-(methylthio)phenyl)-1H-pyrrol-1-yl)ethoxy)phenyl)propanoic acid sodium salt as a white solid. Yield: 0.750 g (80% yield), HPLC Purity: 98.47%, Melting point: 182.4° C.

### Example 24

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

Heat the (S)-2-ethoxy-3-(4-(2-(2-methyl-5-(4-(methylthio)phenyl)-1H-pyrrol-1-yl)ethoxy)phenyl)propanoic acid sodium salt (750 mg, 1.625 mmol) at 170° C. for 6.0 hours. The product was cooled to 30° C. and put overnight at 30° C. Then residue was titruted with diisopropyl ether and dried to yield (S)-2-ethoxy-3-(4-(2-(2-methyl-5-(4-(methylthio)phenyl)-1H-pyrrol-1-yl)ethoxy)phenyl)propanoic acid sodium salt as an off white solid. Yield: 0.600 g (80% yield), HPLC Purity: 91.82%, Melting point: 267.1° C.

### Example 25

### Preparation of (S)-2-ethoxy-3-(4-(2-(2-methyl-5-(4-(methylthio)phenyl)-1H-pyrrol-1-yl)ethoxy)phenyl)propanoic Acid Sodium Salt

To a solution of (S)-2-ethoxy-3-(4-(2-(2-methyl-5-(4-(methylthio)phenyl)-1H-pyrrol-1-yl)ethoxy)phenyl)propanoic acid (0.300 g, 0.682 mmol) in methanol (5.0 ml), Sodium methoxide solution 25 wt. % in methanol (0.156 ml, 0.682 mmol) was added and the reaction mixture was stirred at 30° C. for 48 hours. Reaction mixture was concentrated under reduced pressure and the solid was titruted with diisopropyl ether and solid was dried to yield (S)-2-ethoxy-3-(4-(2-(2-methyl-5-(4-(methylthio)phenyl)-1H-pyrrol-1-yl)ethoxy)phenyl)propanoic acid sodium salt as a white solid. Yield: 0.250 g (79% yield), HPLC Purity: 95.43%.

### Example 26

In 250 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (10.0 g, 0.017 mole) was charged. A solution of Ethyl acetate (72 ml) and water (50 ml) was added at RT under N₂ atm., 50% dil HCl (4.8 ml) solution was added and stirred for 10 minutes. The organic layer was separated and washed with water. The solvent was distilled out using rotavapor under vacuum at 50-55° C., Free acid compound was obtained, In a 250 ml three necked flask, above oily mass (free acid compd.) charged and dissolved in Methanol (50 ml), added Sodium methoxide (0.868 g, 0.016 mole) under nitrogen atmosphere and stirred for 30 minutes at RT. The solvent was distilled out using rotavapor. Solid material was obtained. Triturated the solid material with n-Heptane and stirred for 15 minute. N-Heptane layer was decanted and dissolved the solid again in methanol (40 ml). The solvent was distilled out and dried the solid using rotavapor under vacuum. Yield: 7.9 g, Light brown colored powder, HPLC: Chemical purity: 98.48%, Chiral purity: 84.80%. 72.6° C., amorphous form.

### Example 27

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (500 mg, 1.122 mmol) in 5% NaCl (10 ml), magnesium acetate tetrahydrate (350 mg) was added and the reaction mixture was stirred at 30° C. for 20 hours. Water from the reaction mixture was decanted and then sticky residue was extracted by dichloromethane (2×15 ml). The combined organic layers was washed with water (2×15 ml) and brine (15 ml), dried over sodium sulfate and evaporated under vacuum to yield (S)-2-ethoxy -3-(4-(2-(2-methyl-5-(4-(methylthio)phenyl)-1H-pyrrol-1-yl)ethoxy)phenyl)propanoic acid magnesium salt as a yellow solid. Yield: 531 mg (97.2% yield), HPLC Purity: 92.71%, Melting point: 171.5° C.

### Example 28

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (500 mg, 1.122 mmol) in methanol (10 ml), solution of magnesium acetate tetrahydrate (350 mg) in water (2.0 ml) was added and the reaction mixture was stirred at 30° C. for 20 hours. Reaction mixture was concentrated under reduced pressure and then sticky residue was extracted by dichloromethane (2×15 ml). The combined organic layers was washed with water (2×15 ml) and brine (15 ml), dried over sodium sulfate and evaporated under vacuum to yield (S)-2-ethoxy-3-(4-(2-(2-methyl-5-(4-(methylthio)phenyl)-1H-pyrrol-1-yl)ethoxy)phenyl)propanoic acid magnesium salt as a yellow solid. Yield: 410 mg (73.5% yield), HPLC Purity: 93.08%, Melting point: 179.2° C. HPLC Purity (after 6 months): 90.72%

### Example 29

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (1.0 g, 2.167 mmol) in methanol (20 ml), solution of magnesium chloride (0.103 g, 1.083 mmol) in methanol (4.0 ml) was added and the reaction mixture was stirred at 40-45° C. for 3.0 hours and then stirred overnight at 30° C. for 20 hours. Reaction mixture was filtered, the residue was washed with additional methanol (10 ml) and the filtrate was concentrated under reduced pressure to (S)-2-ethoxy-3-(4-(2-(2-methyl-5-(4-(methylthio)phenyl)-1H-pyrrol-1-yl)ethoxy)phenyl)propanoic acid magnesium salt as white solid.

Yield: 380 mg (37.9% yield), HPLC Purity: 97.68%, Melting point: 154° C.

### Example 30

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (1.0 g, 2.167 mmol) in methanol (20 ml), solution of magnesium acetate tetrahydrate, pure, crystalline (0.232 g, 1.083 mmol) in methanol (4.0 ml) was added and then reaction mixture was heated at 40-45° C. for 3.0 hours and the reaction mixture was stirred overnight at 30° C. for 20 hours. Reaction mixture was filtered and the filtrate was concentrated under reduced pressure to yield (S)-2-ethoxy-3-(4-(2-(2-methyl-5-(4-(methylthio)phenyl)-1H-pyrrol-1-yl)ethoxy)phenyl)propanoic acid magnesium salt as a white solid. Yield: 800 mg (70.5% yield), HPLC Purity: 97.67%

### Example 31

### Preparation of (S)-2-ethoxy-3-(4-(2- (2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid lithium salt (1.0 g, 2.245 mmol) in methanol (20 ml), solution of magnesium acetate tetrahydrate, pure, crystalline (0.241 g, 1.122 mmol) in methanol (4.0 ml) was added and then reaction mixture was heated at 40-45° C. for 3.0 hours and the reaction mixture was stirred overnight at 30° C. for 20 hours. Reaction mixture was filtered and the filtrate was concentrated under reduced pressure to yield (S)-2-ethoxy-3-(4-(2-(2-methyl-5-(4-(methylthio)phenyl)-1H-pyrrol-1-yl)ethoxy)phenyl)propanoic acid magnesium salt as a white solid. Yield: 1.0 g (93% yield), HPLC Purity: 96.95%, Melting point: 260.7° C.,

### Example 32

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid lithium salt (1.0 g, 2.245 mmol) in methanol (20 ml), solution of magnesium chloride (0.107 g, 1.122 mmol) in methanol (4.0 ml) was added and then reaction mixture was heated at 40-45° C. for 3.0 hours and the reaction mixture was stirred overnight at 30° C. for 20 hours. Reaction mixture was filtered and the filtrate was concentrated under reduced pressure to yield (S)-2-ethoxy-3-(4-(2-(2-methyl-5-(4-(methylthio)phenyl)-1H-pyrrol-1-yl)ethoxy)phenyl)propanoic acid magnesium salt as a white solid. Yield: 1.0 g (93% yield), HPLC Purity: 97.10%, Melting point: 137.0° C.

### Example 33

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (1.0 g, 2.167 mmol) in ethanol (20 ml), magnesium chloride (0.103 g, 1.083 mmol) in ethanol (4.0 ml) was added and the reaction mixture was stirred at 40-45° C. for 3.0 hours and then stirred overnight at 30° C. for 20 hours. Separated solid was filtered and the filtrate was evaporated under reduced pressure, the residue was dissolved in ethyl acetate and solid filtered and the filtrate was evaporated under reduced pressure to yield (S)-2-ethoxy-3-(4-(2-(2-methyl-5-(4-(methylthio)phenyl)-1H-pyrrol-1-yl)ethoxy)phenyl)propanoic acid magnesium salt as yellow solid.

Yield: 0.500 g (44.1% yield), HPLC Purity: 96.91%, Melting point: 110.4° C.

### Example 34

### Preparation of (S)-2-ethoxy-3-(4-(242-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid sodium salt (1.0 g, 2.167 mmol) in ethanol (20 ml), magnesium acetate, tetrahydrate (0.232 g, 1.083 mmol) in ethanol (4.0 ml) was added and the reaction mixture was stirred at 40-45° C. for 3.0 hours and then stirred overnight at 30° C. for 20 hours. Separated solid was filtered and the filtrate was concentrated under reduced pressure yield (S)-2-ethoxy-3-(4-(2-(2-methyl-5-(4-(methylthio)phenyl)-1H-pyrrol-1-yl)ethoxy)phenyl)propanoic acid magnesium salt as a white solid. Yield: 0.990 g (79% yield), HPLC Purity: 96.73%, Melting point: 271.6° C.

### Example 35

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

Placed suspension of magnesium (24.88 mg, 1.024 mmol) in methanol (15 ml) into the reaction flask. Then reaction mixture was refluxed at 100° C. for 6.0 hours and then stirred overnight at 30° C. for 2 days. Then solution of (S)-2-ethoxy-3-(4-(2-(2-methyl-5-(4-(methylthio)phenyl)-1H-pyrrol-1-yl)ethoxy)phenyl)propanoic acid (750 mg, 1.706 mmol) in methanol (2.0 ml) was added and the reaction mixture was stirred at to 30° C. for 20 hours. The solvent was evaporated under reduced pressure. Then residue was washed with diethyl ether (20 ml), solid product was filtered and dried to yield (S)-2-ethoxy-3-(4-(2-(2-methyl-5-(4-(methylthio)phenyl)-1H-pyrrol-1-yl)ethoxy)phenyl)propanoic acid magnesium salt as a pale yellow solid. Yield: 0.670 g (85% yield), HPLC Purity: 96.44%, Melting point: 265° C.

### Example 36

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

Placed suspension of magnesium (24.88 mg, 1.024 mmol) in methanol (15 ml) into the reaction flask. Then reaction mixture was refluxed at 100° C. for 6.0 hours and then cooled to 60° C. Then solution of (S)-2-ethoxy-3-(4-(2-(2-methyl-5-(4-(methylthio)phenyl)-1H-pyrrol-1-yl)ethoxy)phenyl)propanoic acid (750 mg, 1.706 mmol) in methanol (2.0 ml) was added and the reaction mixture was stirred at to 100° C. for 30 minute. Then reaction mixture was cooled to 30° C. and then stirred at 30° C. for 48 hours. Then again reaction mixture was heated at 100° C. for 1.0 hours. Then reaction mixture was cooled to 30° C. The solvent was decanted and the residue was washed with methanol (10 ml) and then residue was dried under reduced pressure to yield (S)-2-ethoxy-3-(4-(2-(2-methyl-5-(4-(methylthio)phenyl)-1H-pyrrol-1-yl)ethoxy)phenyl)propanoic acid magnesium salt as a pale yellow solid. Yield: 0.300 g (38% yield), HPLC Purity: 94.57%

### Example 37

### Preparation of (S) 2-Ethoxy-3-(4-{2-[2-methyl-5-(4-methylthiophenyl)-pyrrol-1-yl Ethoxy Phenyl Propionic Acid Cesium Salt

In 50 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (3.0 g, 0.005 mole) was charged. A solution of Ethyl acetate (21 ml) and water (15 ml) was added at RT under N₂, atm., 50% dil HCl (5 ml) solution was added and stirred for 10 minutes. The organic layer was separated and washed with water. The solvent was distilled out using rotavapor under vacuum at 50-55 Free acid compound was obtained. In a 250 ml three necked flask, above oily mass (free acid compd.) charged and dissolved in Methanol (45 ml), added solution of Cesium carbonate (1.57 g, 0.004 mole) in Water (2.1 ml) and stirred for 10 minutes at RT (Hazy solution) and added water (1.5 ml). Stirred for 30 minutes at RT. The solvent was distilled out using rotavapor. Dissolved in DCM (30 ml) and again distilled off Triturated the residue with Hexane (3 times) and decanted and dissolved the residue again in DCM (30 ml). The solvent was distilled out and dried the residue using rotavapor under vacuum. Yield: 3.4 g, Brown colored powder, HPLC: Chemical purity: 98.65%, Chiral purity: 88.10%.

### Example 38

### Preparation of (S) 2-Ethoxy-3-(4-{2-[2-methyl-5-(4-methylthiophenyl)-pyrrol-1-yl Ethoxy Phenyl Propionic Acid Copper Salt

In 250 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (5.0 g, 0.0089 mole) was charged. A solution of Ethyl acetate (100 ml) and water (100 ml) was added at RT under N₂ atm., 50% dil HCl (10 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (80 ml), added solution of Sodium hydroxide (0.464 g, 0.011 mole) in Water (40mL) and stirred for 15 minutes at RT. Added solution of copper (II) acetate hydrate (1.16 g, 0.0058 mole) in Water (40 mL) at 15-20° C. and stirred for 1 hr at 15-20° C. Precipitated solid was filtered and washed with water. Solid was dried in fan dry oven at 45-50° C. Yield: 4.2 g, Light green colored powder, HPLC: Chemical purity: 98.36%, Chiral purity: 88.65%. M.P.: 119.3° C.

### Example 39

### Preparation of (S) 2-Ethoxy-3-(4-{2-[2-methyl-5-(4-methylthiophenyl)-pyrrol-1-yl Ethoxy Phenyl Propionic Acid Cobalt Salt

In 250 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (5.0 g, 0.0089 mole) was charged. A solution of Ethyl acetate (100 ml) and water (100 ml) was added at RT under N₂ atm., 50% dil HCl (10 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (80 ml), added solution of Sodium hydroxide (0.464 g, 0.011 mole) in Water (40 mL) and stirred for 15 minutes at RT. Added solution of cobalt (II) acetate tetrahydrate (1.44 g, 0.0058 mole) in Water (40 mL) at 15-20° C. and stirred for 1 hr at 15-20° C. Precipitated solid was filtered and washed with water. Solid was dried in fan dry oven at 45-50° C. Yield: 4.4 g, Light purple colored powder, HPLC: Chemical purity: 93.17%, Chiral purity: 99.49%. M.P.: 219.4° C.

### Example 40

### Preparation of (S) 2-Ethoxy-3-(4-{2-[2-methyl-5-(4-methylthiophenyl)-pyrrol-1-yl Ethoxy Phenyl Propionic Acid Iron Salt

In 250 ml round bottom flask, (S,S)(-,-)α-methyl benzylamine salt of 2-ethoxy-3-(4-{2-[2-methyl-5-(4-methylthiophenyl)-pyrrol-1-yl]-ethoxy}-phenyl)-propionic acid (5.0 g, 0.0089 mole) was charged. A solution of Ethyl acetate (100 ml) and water (100 ml) was added at RT under N₂ atm., 50% dil HCl (10 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (80 ml), added solution of Sodium hydroxide (0.464 g, 0.011 mole) in Water (40 mL) and stirred for 15 minutes at RT. Added solution of iron (II) sulfate .7H₂O (1.61 g, 0.0058 mole) in Water (40 mL) at 15-20° C., and stirred for 1 hr at 15-20° C. Precipitated solid was filtered and washed with water. Solid was dried in fan dry oven at 45-50° C. Yield: 4.4 g, Brown colored powder, HPLC: Chemical purity: 95.56%, Chiral purity: 99.69%.

### Example 41

### Preparation of (S) 2-Ethoxy-3-(4-{2-[2-methyl-5-(4-methylthiophenyl)-pyrrol-1-yl Ethoxy Phenyl Propionic Acid Manganese Salt

In 250 ml round bottom flask, (S,S)α-methyl benzylamine salt of 2-ethoxy-3-(4-{2-[2-methyl-5-(4-methylthiophenyl)-pyrrol-1-yl]-ethoxy}-phenyl)-propionic acid (6.0 g, 0.01 mole) was charged. A solution of Ethyl acetate (100 ml) and water (100 ml) was added at RT under N₂ atm., 50% dil HCl (12 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (80 ml), added solution of Sodium hydroxide (0.556 g, 0.014 mole) in Water (40 ml) and stirred for 15 minutes at RT. Added solution of manganese (II) acetate tetrahydrate (1.7 g, 0.007 mole) in Water (40 ml) at 15-20° C. and stirred for 1 hr at 15-20° C. Precipitated solid was filtered and washed with water. Solid was dried in fan dry oven at 45-50° C. Yield: 4.9 g, Light grey colored powder, HPLC: Chemical purity: 98.65%, Chiral purity: 86.76%.

### Example 42

### Preparation of (S) 2-Ethoxy-3-(4-{2-[2-methyl-5-(4-1-methylthiophenyl)-pyrrol-1-yl Ethoxy Phenyl Propionic Acid Lead Salt

In 250 ml round bottom flask, (S,S)α-methyl benzylamine salt of 2-ethoxy-3-(4-{2-[2-methyl-5-(4-methylthiophenyl)-pyrrol-1-yl]-ethoxy}-phenyl)-propionic acid (5.0 g, 0.0089 mole) was charged. A solution of Ethyl acetate (45 ml) and water (30 ml) was added at RT under N₂ atm., 50% dil HCl (10 ml) solution was added and stirred for 10 minutes. The organic layer was separated and washed with water. The solvent was distilled out using rotavapor under vacuum at 50-55° C. Free acid compound was obtained. In a 250 ml three necked flask, above oily mass (free acid compd.) charged and dissolved in Methanol (25 ml), added solution of Sodium hydroxide (0.465 g, 0.011 mole) in Water (25 mL) and stirred for 15 minutes at RT. Added. solution of Lead acetate trihydrate (2.2 g, 0.0058 mole) in Methanol (10 mL) at 15-20° C. and added Water (7 ml). Stirred for 45 min at 15-20° C. Precipitated solid was filtered and washed with water. Solid was dried in fan dry oven at 45-50° C. Yield: 5.0 g, Brown colored powder, HPLC: Chemical purity: 97.82%, Chiral purity: 87.28%, 71.2° C.

### Example 43

### Preparation of Pre-Mix of (S) 2-Ethoxy-3-(4-{2-[2-methyl-5-(4-methylthiophenyl)-pyrrol-1-yl Ethoxy Phenyl Propionic Acid and Copovidone

(S)-2-ethoxy-3-(4-(2-(2-methyl-5-(4-(methylthio)phenyl)-1H-pyrrol-1 yl)ethoxy)phenyl)propanoic acid (1 g) was dissolved in dichloromethane (50 mL), added Copovidone 90 (2 g) to the this solution and the resulting solution was stirred at 30-35° C. till clear solution is obtained. The resulting solution is passed over the bed of Hyflo. The solvents of filtrate were removed on a rotatory evaporator under reduced pressure, keeping the bath temperature at 50-55° C. Added methanol (150 mL) to the residue and solvents were removed again on a rotatory evaporator under reduced pressure, keeping the bath temperature 50-55 OC. The solid obtained was powdered on a ball mill to get, off white solid (608 mg) pre-mix of (S) 2-Ethoxy-3-(4-{2-[2-methyl-5-(4-methylthiophenyl)-pyrrol-1-yl ethoxy phenyl propionic acid and Copovidone

### Example 44

### Preparation of Pre-Mix of Magnesium (S) 2-Ethoxy-3-(4-{2-[2-methyl-5-(4-methylthiophenyl)-pyrrol-1-yl Ethoxy Phenyl Propionate and HPMC

Magnesium (S)-2-ethoxy-3-(4-(2-(2-methyl-5-(4-(methylthio)phenyl)-1H-pyrrol-1-yl)ethoxy)phenyl)propionate (1 g) and added HPMC (2 g) to the this. The solid mixture was dissolved in dichloromethane (100 mL) and methanol (100 mL). The resulting solution was stirred at 40-45° C. till clear solution is obtained. The resulting solution is passed over the bed of Hyflo. The solvents of filtrate were removed on a rotatory evaporator under reduced pressure, keeping the bath temperature at 50-55° C. The solvents were removed on a rotatory evaporator under reduced pressure, keeping the bath temperature 50-55° C. The solid obtained was powdered on a ball mill to get, off white solid (1.5 g) pre-mix of Magnesium (S)-2-ethoxy-3-(4-(2-(2-methyl-5-(4-(methylthio)phenyl)-1H-pyrrol-1-yl)ethoxy)phenyl)propionate and HPMC.

### Example 45

### Preparation of Pre-Mix of Magnesium (S) 2-Ethoxy-3-(4-{2-[2-methyl-5-(4-methylthiophenyl)-pyrrol-1-yl Ethoxy Phenyl Propionate and HPMC (1:3)

Similarly, pre-mix saroglitazar magnesium and HPMC in ratio of 1:3 is prepared as per process disclosed in Example 45.

### Example 46

### Preparation of Pre-Mix of Magnesium (S) 2-Ethoxy-3-(4-{2-[2-methyl-5-(4-methylthiophenyl)-pyrrol-1-yl Ethoxy Phenyl Propionate and HPMC (1:4)

Similarly, pre-mix Saroglitazar magnesium and HPMC in ratio of 1:4 is prepared as per process disclosed in Example 45.

### Example 47

### Preparation of Pre-Mix of Magnesium (S) 2-Ethoxy-3-(4-{2-[2-methyl-5-(4-methylthiophenyl)-pyrrol-1-yl Ethoxy Phenyl Propionate and HPMC (1:5)

Similarly, pre-mix Saroglitazar magnesium and. HPMC in ratio of 1:5 is prepared as per process disclosed in Example 45.

### Example 48

### Preparation of Pre-Mix of Magnesium (S) 2-Ethoxy-3-(4-(2-[2-methyl-5-(4-methylthiophenyl)-pyrrol-1-yl Ethoxy Phenyl Propionic Acid and Methacrylic Acid (1:2)

Magnesium (S)-2-ethoxy-3-(4-(2-(2-methyl-5-(4-(methylthio)phenyl)-1H-pyrrol-1-yl)ethoxy)phenyl)propionic acid (1 g) was dissolved in mixture of dichloromethane (50 mL) and methanol (50 mL), added Eudragit (2 g) to the this solution and the resulting solution was stirred at 30-35° C. till clear solution is obtained. The resulting solution is passed over the bed of Hyflo.

The solvents of filtrate were removed on a rotatory evaporator under reduced pressure, keeping the bath temperature at 50-55° C. Added methanol (100 mL) to the residue and solvents were removed again on a rotatory evaporator under reduced pressure, keeping the bath temperature 50-55° C. The solid obtained was powdered on a ball mill to get, off white solid (1.6 g) pre-mix of magnesium (S)-2-ethoxy-3-(4-(2-(2-methyl-5-(4-(methylthio)phenyl)-1H-pyrrol-1-yl)ethoxy)phenyl)propionic acid and Eudragit.

### Example 49

### Preparation of Pre-Mix of Magnesium (S) 2-Ethoxy-3-(4-{2-[2-methyl-5-(4-methylthiophenyl)-pyrrol-1-yl Ethoxy Phenyl Propionic Acid and Methacrylic Acid (1:3)

Similarly, pre-mix saroglitazar magnesium and Methacrylic acid in ratio of 1:3 is prepared as per process disclosed in Example 48.

### Example 50

### Preparation of Pre-Mix of Magnesium (S) 2-Ethoxy-3-(4-{2-[2-methyl-5-(4-methylthiophenyl)-pyrrol-1-yl Ethoxy Phenyl Propionic Acid and Methacrylic Acid (1:4)

Similarly, pre-mix Saroglitazar magnesium and Methacrylic acid in ratio of 1:4 is prepared as per process disclosed in Example 48.

### Example 51

### Preparation of Pre-Mix of Magnesium (S) 2-Ethoxy-3-(4-{2-[2-methyl-5-(4-methylthiophenyl)-pyrrol-1-yl Ethoxy Phenyl Propionic Acid and Methacrylic Acid (1:5)

Similarly, pre-mix saroglitazar magnesium and Methacrylic acid in ratio of 1:5 is prepared as per process disclosed in Example 48.

### Example—52

### Preparation of Crystalline (S) 2-Ethoxy-3-(4-{2[2-methyl-5-(4-methylthiophenyl)-pyrrol-1-yl Ethoxy Phenyl Propionic Acid Metformin Salt

(S)-2-ethoxy-3-(4-(2-(2-methyl-5-(4-(methylthio)pheny)-1H-pyrrol-1 yl)ethoxy)phenyl)propanoic acid (8.6 g, 19.564 mmol) was dissolved in methanol (50 mL). To the this solution was added solution of metformin base (2.5 g, 19.564 mmol) in 15 ml methanol and the resulting mixture was stirred and refluxed over a period of 19 h. The resulting solution was filtered over a bed of Hyflo. The solvents were removed on a rotatory evaporator under reduced pressure to get sticky solid. To the sticky solid added ethyl acetate (70 mL) and stirred at 27-30 OC over a period of 2 h to get solid. The solid was filtered, washed with ethyl acetate dried and air dried to afford off white solid (3 g) as (S) 2-Ethoxy-3-(4-{2-[2-methyl-5-(4-methylthiophenyl)-pyrrol-1-yl ethoxy phenyl propionic acid Metformin salt.

### Example 53

### Preparation of Pre-Mix of (S) 2-Ethoxy-3-(4-{2-[2-methyl-5-methylthiophenyl)-pyrrol-1-yl Ethoxy Phenyl Propionic Acid and Magnesium Aluminoametasilcate (Neusilin)

((S)-2-ethoxy-3-(4-(2-(2-methyl-5-(4-(methylthio)phenyl)-1H-pyrrol-1-yl)ethoxy)phenyl)propanoic acid (3 g, 6.82 mmol) in ethyl acetate (30 mL) was placed in a round bottom flask. Neusilin US2 (1.8 g) was added slowly wider stirring at 25-30° C. for 1 min.

Reaction suspension was stirred at 25-30° C. over a period of 10 min. Reaction mixture was concentrated on rotatory evaporator to remove ethyl acetate at 30° C. to get off white solid. The solid obtained was passed through 300 mesh size sieve to get fine powder of Saroglitazar acid with Neusilin US2. Yield—3.0 g. GC (for residual solvent ethyl acetate found 10.89 ppm within limit), UPLC (97.87%) Assay (58.25%).

Following examples were synthesized using same procedure as provided in example 48 using appropriate reagents. Analytical data of each examples are given below

### Example 54

### Preparation of Pre-Mixture of (S)-2-ethoxy-3-(4-(2-(2-methyl-5-(4-(methylthio)phenyl)-1H-pyrrol-1-yl)ethoxy)phenyl)propanoic Acid with Magnesium Aluminometasilicate (Neusilin) (1:1)

UPLC: 97.87%, After 6 month UPLC purity: 91.61%, GC (for residual solvent) ethyl acetate—12 ppm, when stored at 2-8° C.

### Example 55

### Pre-Mixture of (S)-2-ethoxy-3-(4-(2-(2-methyl-5-(4-(methylthio)phenyl)-1H-pyrrol-1-yl)ethoxy)phenyl)propanoic Acid with Copovidone 60 (1:3)

UPLC: 95%, GC (for residual solvent) Methanol-27 ppm, when stored at 2-8° C.

### Example 56

### Pre-Mixture of (S)-2-ethoxy-3-(4-(2-(2-methyl-5-(4-(methylthio)phenyl)-1H-pyrrol-1-yl)ethoxy)phenyl)propanoic Acid Copovidone 90 (1:5)

UPLC: 95.7%, GC (for residual solvent) Dichloromethane-158 ppm, ethylacetate-33 ppm.

### Example 57

### Pre-Mixture of (S)-2-ethoxy-3-(4-(2-(2-methyl-5-(4-(methylthio)phenyl)-1H-pyrrol-1-yl)ethoxy)phenyl) Propanoic Acid and Copovidone 90 (1:1.2)

UPLC: 95.74%, GC (for residual solvent) Dichloromethane-Nil, ethylacetate-12 ppm.

### Example 58

### Pre-Mixture of (S)-2-ethoxy-3-(4-(2-(2-methyl-5-(4-(methylthio)phenyl)-1H-pyrrol-1-yl)ethoxy)phenyl) Propanoic Acid with Copovidone (90) (1:1)

Analytical data: UPLC: 86.69%, after two month UPLC: 73.22%, when stored at 2-8° C.

### Example 59

### Pre-Mixture of (S)-2-ethoxy-3-(4-(2-(2-methyl-5-(4-(methylthio)phenyl)-1H-pyrrol-1-yl)ethoxy)phenyl)propanoic Acid with HPMC.AS (1:1.2)

UPLC 73%

### Example 60

### Pre-Mixture of (S)-2-ethoxy-3-(4-(2-(2-methyl-5-(4-(methylthio)phenyl)-1H-pyrrol-1-yl)ethoxy)phenyl)propanoic Acid with HPMC.AS (1:2)

UPLC: 87.75%

### Example 61

### Pre-Mixture of (S)-2-ethoxy-3-(4-(2-(2-methyl-5-(4-(methylthio)phenyl)-1H-pyrrol-1-yl)ethoxy)phenyl)propanoic Acid with HPMC.AS (1:3)

UPLC: 90.31%

### Example 62

### Pre-Mixture of (S)-2-ethoxy-3-(4-(2-(2-methyl-5-(4-(methylthio)phenyl)-1H-pyrrol-1-yl)ethoxy)phenyl)propanoic Acid with HPMC.AS (1:5)

UPLC: 89.80%

### Example 63

### Pre-Mixture of (S)-2-ethoxy-3-(4-(2-(2-methyl-5-(4-(methylthio)phenyl)-1H-pyrrol-1-yl)ethoxy)phenyl)propanoic Acid with Diatomaceous Earth (1:4)

UPLC: 92.05%, GC (for residual solvent) Methanol Not detected, ethylacetate-99 ppm

### Example 64

### Pre-Mixture of (S)-2-ethoxy-3-(4-(2-(2-methyl-5-(4-(methylthio)phenyl)-1H-pyrrol-1-yl)ethoxy)phenyl)propanoic Acid with Zeolite (1:3)

UPLC: 91.25%, GC (for residual solvent) Methanol Not detected, ethylacetate-77 ppm

### Example 65

### Pre-Mixture of (S)-2-ethoxy-3-(4-(2-(2-methyl-5-(4-(methylthio)phenyl)-1H-pyrrol-1-yl)ethoxy)phenyl)propanoic Acid with Tribasic Calcium Phosphate (1:4)

UPLC: 88.18%, GC (for residual solvent) Methanol Not detected, Ethyl acetate 857 ppm

### Example 66

### Pre-Mixture of (S)-2-ethoxy-3-(4-(2-(2-methyl-5-(4-(methylthio)phenyl)-1H-pyrrol-1-yl)ethoxy)phenyl)propanoic Acid with Tungstic Acid (1:4)

UPLC: 91.61%, GC (for residual solvent) Methanol Not detected, Ethyl acetate 4281 ppm

### Example 67

### Pre-Mixture of (S)-2-ethoxy-3-(4-(2-(2-methyl-5-(4-(methylthio)phenyl)-1H-pyrrol-1-yl)ethoxy)phenyl)propanoic Acid with Methacrylic Acid (1:2)

UPLC: 92.06%, UPLC after 2 month 81.12%, UPLC after 9 month 69.72% GC (for residual solvent), Dichloromethane-Not detected, when stored at 2-8° C.

### Example 68

### Pre-Mixture of (S)-2-ethoxy-3-(4-(2-(2-methyl-5-(4-(methylthio)phenyl)-1H-pyrrol-1-yl)ethoxy)phenyl)propanoic Acid with Methacrylic Acid (1:3)

UPLC: 91.98%, UPLC after 2 month 79.74%, UPLC after 9 month 68.59%, when stored at 2-8° C.

### Example 69

### Pre-Mixture of (S)-2-ethoxy-3-(4-(2-(2-methyl-5-(4-(methylthio)phenyl)-1H-pyrrol-1-yl)ethoxy)phenyl)propanoic Acid with Methacrylic Acid (1:4)

UPLC: 91.41%, UPLC after 2 month 82.45%, UPLC after 9 month 72.18% GC (for residual solvent), Dichloromethane-Not detected, when stored at 2-8° C.

### Example 70

### Pre-Mixture of (S)-2-ethoxy-3-(4-(2-(2-methyl-5-(4-(methylthio)phenyl-1H-pyrrol-1-yl)ethoxy)phenyl)propanoic Acid with Methacrylic Acid (1:5)

UPLC: 92.05%, UPLC after 2 months 81.81%, UPLC after 9 month 71.22%, when stored at 2-8° C.

### Example 71

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

To a solution of (S)-2-ethoxy-3-(4-(2-(2-methyl-5-(4-(methylthio)phenyl)-1H-pyrrol-1-yl)ethoxy)phenyl)propanoic acid (0.900 g, 2.047 mmol) in methanol 1.15 ml), eq. ammonium hydroxide (0.4 ml) was added and the stirred the reaction mixture for 1.0 hour at 30° C. Then in this magnesium sulfate heptahydrate c.p. (0.303 g, 1.228 mmol) was added and the reaction mixture was stirred at 30° C. for 30 hours. The solvent was evaporated under reduced pressure. Then residue was washed with water (20 ml), then residue was titruted with diethyl ether and solvent decanted and the product was filtered under reduced pressure to yield thick gummy product. Then product was air dried for 2 days to yield yellow solid. Yield: 0.670 g (79% yield) HPLC Purity: 95.0%, Melting point: 269.8° C.

### Example 72

### Preparation of (S)-2-ethoxy-3-(4-(2-(2-methyl-5-(4-(methylthio)phenyl)-1H-pyrrol-1-yl)ethoxy)phenyl)propanoic Acid Magnesium Salt

(S)-2-ethoxy-3-(4-(2-(2-methyl-5-(4-(methylthio)phenyl)-1H-pyrrol-1-yl)ethoxy)phenyl)propanoic acid magnesium salt (400 mg, 0.864 mmol) was heated at 100° C. for 24 hours to yield (S)-2-ethoxy-3-(4-(2-(2-methyl-5-(4-(methylthio)phenyl)-1H-pyrrol-1-yl)ethoxy)phenyl)propanoic acid magnesium salt as a yellow solid.

### Yield: 0.310 g (78% HPLC Purity: 93.79%.

### Stability

Stability of selected compounds was measured by HPLC purity (as given below in Table 1) shows these compounds have shown stability for 6 months.

TABLE 1

| | | HPLC (% | HPLC (% |
|---|---|---|---|
| | | purity) | purity) |
| Example | Compound | At 0 month) | After 6 months |
| 12 | S)-2-ethoxy-3-(4-(2-(2- | 96.82% | 95.50% |
| | methyl-5-(4- | | |
| | (methylthio)phenyl)-1H- | | |
| | pyrrol-1- | | |
| | yl)ethoxy)phenyl)propanoic | | |
| | acid lithium salt | | |
| 14 | (S)-2-ethoxy-3-(4-(2-(2- | 96.79% | 94.94% |
| | methyl-5-(4- | | |
| | (methylthio)phenyl)-1H- | | |
| | pyrrol-1- | | |
| | yl)ethoxy)phenyl)propanoic | | |
| | acid aluminum salt | | |
| 16 | amorphous form of (S)-2- | 96.83% | 96.76% |
| | ethoxy-3-(4-(2-(2-methyl- | | |
| | 5-(4-(methylthio)phenyl)- | | |
| | 1H-pyrrol-1- | | |
| | yl)ethoxy)phenyl)propanoic | | |
| | acid calcium salt | | |
| 28 | (S)-2-ethoxy-3-(4-(2-(2- | 93.08% | 90.72% |
| | methyl-5-(4- | | |
| | (methylthio)phenyl)-1H- | | |
| | pyrrol-1- | | |
| | yl)ethoxy)phenyl)propanoic | | |
| | acid magnesium salt | | |
| 29 | (S)-2-ethoxy-3-(4-(2-(2- | 97.68% | 94.63% |
| | methyl-5-(4- | | |
| | (methylthio)phenyl)-1H- | | |
| | pyrrol-1- | | |
| | yl)ethoxy)phenyl)propanoic | | |
| | acid magnesium salt | | |
| 31 | (S)-2-ethoxy-3-(4-(2-(2- | 96.95% | 95.55% |
| | methyl-5-(4- | | |
| | (methylthio)phenyl)-1H- | | |
| | pyrrol-1- | | |
| | yl)ethoxy)phenyl)propanoic | | |
| | acid magnesium salt | | |
| 32 | (S)-2-ethoxy-3-(4-(2-(2- | 97.10% | 95.78% |
| | methyl-5-(4- | | |
| | (methylthio)phenyl)-1H- | | |
| | pyrrol-1- | | |
| | yl)ethoxy)phenyl)propanoic | | |
| | acid magnesium salt | | |

### Pharmacological Data

### Comparative Efficacy Study (Triglyceride Lowering Effects) of Various Salts of Saroglitazar and Formula (I) in Swiss Albino Mice

The in-vivo efficacy of test compound was evaluated in Swiss albino mice. Anti-dyslipidemic drugs have been reported to lower circulating levels of triglyceride in Swiss albino mice through their effect on genes involved in the peroxisomal fatty acid beta oxidation via PPAR alpha agonism. Therefore, this species is preferred for evaluation of their efficacy in lowering circulating triglyceride (TG) levels.

In this experiment, ten to eleven week old male Swiss albino mice were issued and kept for acclimatization. Near the end of the acclimatization period, animals judged to be suitable for testing were bled under light anesthesia and serum samples were analyzed for serum triglyceride levels. Animals were selected according to triglyceride levels in the range of 42-102 mg/dl and divided into various treatment groups (Table no. 1) of 6 animals each such that the average TG levels of animals in each group were not significantly different from the others.

TABLE NO. 2
Treatment groups and dose levels

| | | | | Number |
|---|---|---|---|---|
| | | | | of |
| Group | Example No | Treatment | Dose (mg/kg) | Animals |
| 1 | — | Vehicle Control (MiliQ water) | 0 mg/kg | 6 |
| 2 | — | Saroglitazar (formula (I)) | 1 mg/kg | 6 |
| 3 | 14 | Aluminium salt of Saroglitazar 1.18 mg/kg, | 1 mg/kg | 6 |
| | | p.o. equivalent to 1 mg/kg of | | |
| | | corresponding free acid | | |
| 4 | 16 | Calcium salt of Saroglitazar at 1.18 mg/kg | 1 mg/kg | 6 |
| | | equivalent to 1 mg/kg of corresponding | | |
| | | free acid | | |
| 5 | 6 | Sodium salt of Saroglitazar at 1.05 mg/kg | 1 mg/kg | 6 |
| | | equivalent to 1 mg/kg of corresponding | | |
| | | free acid | | |
| 6 | 32 | Magnesium salt of Saroglitazar at 1.11 | 1 mg/kg | 6 |
| | | mg/kg equivalent to 1 mg/kg of | | |
| | | corresponding free acid | | |
| 7 | 29 | Magnesium salt of Saroglitazar at 1.11 | 1 mg/kg | 6 |
| | | mg/kg equivalent to 1 mg/kg of | | |
| | | corresponding free acid | | |
| 8 | 28 | Magnesium salt of Saroglitazar at 1.11 | 1 mg/kg | 6 |
| | | mg/kg equivalent to 1 mg/kg of | | |
| | | corresponding free acid | | |

*p < 0.05,
**p < 0.01 and
***p < 0.001 vs Vehicle control (One way ANOVA)
#p < 0.05 vs formula(I) (t-test)

TABLE 3
Effect on serum triglyceride levels

% Change

| Example | | Serum triglycerides (mg/dl) | | Day 6 vs | % Change |
|---|---|---|---|---|---|
| No. | Compound | Day 0 | Day 6 | Day 0 | Vs control |
| — | Vehicle | 59.3 ± 5.1 | 104.4 ± 14.7 | 79.9 ± 28.6 | |
| | Control | | | | |
| | (MiliQ water) | | | | |
| — | Saroglitazar | 61.8 ± 7.4 | 65.9 ± 7.7* | 8.7 ± 9.0 | −38.2 ± 5.1 |
| | (formula (I)) | | | | |
| 14 | Aluminium | 61.9 ± 7.4 | 37.4 ± 3.4***# | −35.1 ± 10.9 | −63.1 ± 6.2 |
| | salt of | | | | |
| | Saroglitazar | | | | |
| | 1.18 mg/kg, | | | | |
| | p.o. | | | | |
| | equivalent to | | | | |
| | 1 mg/kg of | | | | |
| | corresponding | | | | |
| | free acid | | | | |
| 16 | Calcium salt | 61.3 ± 6.4 | 38.2 ± 5.6***# | −33.9 ± 14.3 | −62.4 ± 8.1 |
| | of | | | | |
| | Saroglitazar | | | | |
| | at 1.18 mg/kg | | | | |
| | equivalent to | | | | |
| | 1 mg/kg of | | | | |
| | corresponding | | | | |
| | free acid | | | | |
| 6 | Sodium salt | 59.6 ± 5.4 | 39.0 ± 7.5***# | −34.4 ± 10.5 | −62.7 ± 6.0 |
| | of | | | | |
| | Saroglitazar | | | | |
| | at 1.05 mg/kg | | | | |
| | equivalent to | | | | |
| | 1 mg/kg of | | | | |
| | corresponding | | | | |
| | free acid | | | | |
| 32 | Magnesium | 61.9 ± 7.7 | 32.6 ± 4.9***# | −44.5 ± 10.4 | −68.4 ± 5.9 |
| | salt of | | | | |
| | Saroglitazar | | | | |
| | at 1.11 mg/kg | | | | |
| | equivalent to | | | | |
| | 1 mg/kg of | | | | |
| | corresponding | | | | |
| | free acid | | | | |
| 29 | Magnesium | 59.4 ± 5.0 | 37.0 ± 3.5***# | −35.5 ± 7.5 | −63.3 ± 4.3 |
| | salt of | | | | |
| | Saroglitazar | | | | |
| | at 1.11 mg/kg | | | | |
| | equivalent to | | | | |
| | 1 mg/kg of | | | | |
| | corresponding | | | | |
| | free acid | | | | |
| 28 | Magnesium | 64.3 ± 8.6 | 30.0 ± 3.8***# | −47.7 ± 10.2 | −70.2 ± 5.8 |
| | salt of | | | | |
| | Saroglitazar | | | | |
| | at 1.11 mg/kg | | | | |
| | equivalent to | | | | |
| | 1 mg/kg of | | | | |
| | corresponding | | | | |
| | free acid | | | | |

Test compounds were formulated at specified doses in vehicle (MiliQ Water). The animals were dosed orally, once daily in the morning during six days, starting from next day of grouping with vehicle or test compound. The animals were weighed prior to dosing, and based on these weights; the volume of administration was calculated. The volume of formulation administered to each mouse was 10 ml/kg body weight.

On day 6, one-hr after the dose administration, blood (0.25 ml) was collected from retro-orbital sinus of the anaesthetized animals. Serum was separated by centrifugation. Serum was analyzed for triglyceride levels. Analysis for serum triglyceride levels was performed using Spectrophotometer and commercially available kit. Calculations for determination of % change and % reduction in serum TG levels (Table 3) were performed using MS Excels sheet.

### CONCLUSION

Six days oral administration of formula (I) at 1 mg/kg, p.o, results in 38.2% reduction in serum triglyceride level. Six days treatment with aluminium salt of saroglitazar, calcium salt of saroglitazar, sodium salt, magnesium salts of saroglitazar, at a dose of 1 mg/kg, p.o. showed significant 63,1%, 62.4%, 62.7%, 68.4%, 63.3% and 70.2% reduction respectively, in serum triglyceride levels as compared to vehicle control group.

We claim:

1. Saroglitazar sodium in at least partially crystalline form.

2. Saroglitazar sodium as claimed in claim 1 having a crystalline purity in range of 10-90%, preferably crystalline purity 10-80%, or crystalline purity of at least 10-70%, or crystalline purity of at least 10-60%, or crystalline purity of at least 10-50%, or crystalline purity of at least 10-40%, or crystalline purity of at least 10-30%, or crystalline purity of at least 10-20% and including all values in between the defined ranges.

3. Saroglitazar sodium as claimed in claim 1, selected from
a) compound having PXRD peaks at 4.567, 8.006, 9.219±0.2 degrees 2-theta or a PXRD pattern as per FIG. 18 or a melting point 243.8° C.;
b) compound having a PXRD peaks at 4.632, 8.053, 9.286±0.2 degrees 2-theta or a PXRD pattern as per FIG. 19;
c) compound having a Powder XRD peaks at 4.643, 8.044, 9.277, 12.269, 13.381, 14.860±0.2 degrees 2-theta or a PXRD pattern as per FIG. 20 or a melting point of 240.6° C.;
d) compound having a Powder XRD peaks at 4.513, 7.918, 9.147, 13.129, 14.678, 15.385±0.2 degrees 2-theta or a PXRD pattern as per FIG. 22 or a melting point of 182.4° C.;
e) compound having Powder XRD peaks at 3.158, 4.552, 7.873, 9.102, 3.131, 14.655, 5.363, 17.447±0.2 degrees 2-theta or a PXRD pattern as per FIG. 23 or a melting point 267.1° C.

4. The saroglitazar sodium as claimed in claim 1, wherein the saroglitazar sodium is in crystalline form.

5. The Crystalline saroglitazar sodium as claimed in claim 4, wherein the saroglitazar sodium is selected from
a) Compounds having a powder X-ray diffraction pattern having peaks at about 4.628, 8.043, 9.288±0.2 degrees 2-theta or a PXRD pattern as per FIG. 4 or a melting point 238.3° C.
b) Compounds having a powder X-ray diffraction pattern having peaks at about 4.605, 8.019, 9.254±0.2 degrees 2-theta or a PXRD pattern as per FIG. 5 or melting point 263.2° C.;
c) Compounds having a powder X-ray diffraction pattern having additional peaks at about 13.288, 14.816, 15.502, 16.912, 21.276±0.2 degrees 2-theta or a PXRD pattern as per FIG. 6 or a melting point 240.6° C.;
d) Compounds having a powder X-ray diffraction pattern having peaks at about 3.864, 7.952, 8.225, 16.195, 18.663±0.2 degrees 2-theta or a PXRD pattern as per FIG. 24.

6. The saroglitazar sodium as claimed in claim 1, wherein saroglitazar sodium is in amorphous form.

7. Amorphous saroglitazar sodium as claimed in claim 6, wherein saroglitazar sodium has
a) PXRD pattern as per FIG. 21 or a melting point 139.1° C.;
b) PXRD pattern as per FIG. 25 or a melting point 72.6° C.

8. Saroglitazar magnesium in at least partially crystalline form.

9. Saroglitazar magnesium as claimed in claim 8 having a crystalline purity in range of 10-90%, preferably crystalline purity 10-80%, or crystalline purity of at least 10-70%, or crystalline purity of at least 10-60%, or crystalline purity of at least 10-50%, or crystalline purity of at least 10-40%, or crystalline purity of at least 10-30%, or crystalline purity of at least 10-20% and including all values in between the defined ranges

10. The saroglitazar magnesium as claimed in claim 8, selected from
a) Compounds having powder XRD peaks at 4.177, 19.029, 23.038, 32.092, 33.818±0.2 degrees 2-theta or a PXRD pattern as per FIG. 26 or melting point of 171.5° C.;
b) Compounds having powder XRD peaks at 4.122, 19.033, 28.037, 29.009, 32.121, 33.870±0.2 degrees 2-theta or PXRD pattern as per FIG. 27 or melting point 179.2° C.;
c) Compounds having powder XRD peaks at 4.346, 9.584, 12.579, 20.803±0.2 degrees 2-theta or PXRD pattern as per FIG. 30 or melting point at 260.7° C.;
d) Compounds having powder XRD peaks at 4.538, 7.916, 9.134, 31.650±0.2 degrees 2-theta or PXRD pattern as per FIG. 31 or melting point at 137.0° C.;
e) Compounds having powder XRD peaks at 7.935, 4.533, 29.933, 34.228, 35.057±0.2 degrees 2-theta or PXRD pattern as per FIG. 47 and melting point at 269.8° C.;
f) Compounds having powder XRD peaks at 4.572, 7.386, 7.990, 9.248, 29.957, 35.116, 37.802±0.2 degrees 2-theta or PXRD pattern as per FIG. 48.

11. The saroglitazar magnesium as claimed in claim 8, wherein the saroglitazar magnesium is in crystalline form.

12. The crystalline saroglitazar magnesium as claimed in claim 11, selected from
a) Compounds having powder XRD peaks at 3.980, 27.350, 31.685±0.2 degrees 2-theta or a PXRD pattern as per FIG. 7 or a melting point at 148.4° C.;
b) Compounds having powder XRD peaks at about 4.418, 8.809, 20.074±0.2 degrees 2-theta or a PXRD pattern as per FIG. 8 and melting point 223° C.;
c) Compounds having powder XRD peaks at 4.488, 7.896, 8.979±0.2 degrees 2-theta or a PXRD pattern as per FIG. 9 or melting point at 185-190° C.

13. The saroglitazar magnesium as claimed in claim 8, wherein the saroglitazar magnesium is in amorphous form.

14. Amorphous saroglitazar magnesium as claimed in claim 13 selected from
a) Compounds having PXRD pattern as per FIG. 10 or melting point of 219° C.;
b) Compounds having PXRD pattern as per FIG. 28 or melting point of 154° C.;
c) Compounds having PXRD pattern as per FIG. 29;
d) Compounds having PXRD pattern as per FIG. 32 or melting point of 110.4° C.;
e) Compounds having PXRD pattern as per FIG. 33 or melting point of 271.6° C.;
f) Compounds having PXRD pattern as per FIG. 34 or melting point of 265.1° C.;
g) Compounds having PXRD pattern as per FIG. 35.

15. Amorphous saroglitazar salts selected from lithium, potassium and calcium salts.

16. Amorphous saroglitazar lithium salt as claimed in claim 15, selected from
a) Compounds having PXRD pattern as per FIG. 1 or melting point 131.2° C.;
b) Compounds having PXRD pattern as per FIG. 12 or melting point 285.5° C.

17. Amorphous saroglitazar potassium as claimed in claim 16, wherein saroglitazar potassium has
a) PXRD pattern as per FIG. 2 and melting point 62.1° C.;
b) PXRD pattern as per FIG. 13.

18. Amorphous saroglitazar calcium as claimed in claim 15, selected from
a) Compounds having PXRD pattern as per FIG. 3 or a melting point of 197.8° C.;
b) Compounds having PXRD pattern as per FIG. 15 or a melting point of 214.6° C.;
c) Compounds having PXRD pattern as per FIG. 16 or a melting point of 175.2° C.;
d) Compounds having PXRD pattern as per FIG. 17 or a melting point of 150.6° C.

19. Novel saroglitazar salts of formula (Ia) 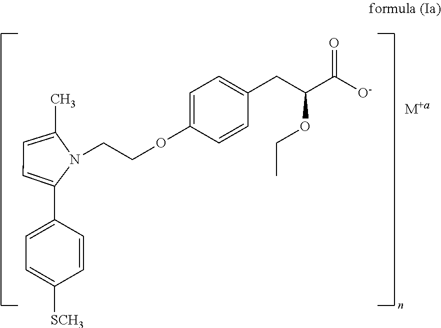
Wherein M^{+a} is cation selected from cesium, copper, cobalt, iron, manganese, lead, aluminum, metformin-1-glutamic acid; n is integer selected from 1, 2, 3.

20. Novel salts of saroglitazar as claimed in claim 19, wherein each salt is at least partially crystalline, crystalline or in amorphous forms.

21. Novel premixes of saroglitazar or its pharmaceutically acceptable salts with suitable pharmaceutically acceptable excipients or suitable secondary therapeutic agents.

22. Novel premixes of saroglitazar and its pharmaceutically acceptable salts as claimed in claim 21 wherein pharmaceutically acceptable excipients are selected from magnesium aluminometasilicate (neusilin), copovidone, HPMC, HPMC.AS, Methacrylic acid (eudragit), diatomaceous earth, metformin, zeolite, tungstic acid, tribasic calcium phosphate.

23. Novel premixes of saroglitazar and its pharmaceutically acceptable salts with pharmaceutically acceptable excipients as claimed in claim 21 and secondary therapeutic agents wherein each premix is at least partially crystalline, crystalline or amorphous forms.

24. Novel premixes of Saroglitazar with suitable excipients selected from
a) Saroglitazar and magnesium aluminometasilicate;
b) Saroglitazar and copovidone;
c) Saroglitazar magnesium and HPMC;
d) Saroglitazar and HPMC.AS;
e) Saroglitazar magnesium and Methacrylic acid;
f) Saroglitazar and metformin;
g) Saroglitazar and tribasic calcium phosphate;
h) Saroglitazar and tungstic acid

25. Novel premixes of saroglitazar and its pharmaceutically acceptable salts as claimed in claim 24, wherein the ratio of ingredients are
a) Ratio of Saroglitazar: magnesium aluminometasilicate is selected from 1:0.6 and 1:1;
b) Ratio of Saroglitazar: copovidone is selected from 1:1, 1:1.2, 1:2, 1:3, 1:5;
c) Ratio of Saroglitazar magnesium: HPMC is selected from 1:2, 1:3, 1:4, 1:5;
d) Ratio of Saroglitazar: HPMC.AS is selected from 1:1.2, 1:2, 1:3, 1:5;
e) Ratio of Saroglitazar magnesium: Methacrylic acid is selected from 1:2, 1:3, 1:4, 1:5;
f) Ratio of Saroglitazar: Methacrylic acid is selected from 1:2, 1:3, 1:4, 1:5;
g) Ratio of Saroglitazar: metformin is 1:1;
h) Ratio of Saroglitazar: diatomaceous earth is 1:4;
i) Ratio of Saroglitazar: zeolite is 1:3;
j) Ratio of Saroglitazar: tribasic calcium phosphate is 1:4;
k) Ratio of Saroglitazar: tungstic acid is 1:4.

26. Novel premixes of saroglitazar and its pharmaceutically acceptable salts as claimed in claim 24 wherein
a) Ratio of Saroglitazar: magnesium aluminometasilicate is 1:0.6 and the premix having PXRD pattern as per FIG. 46;
b) Ratio of Saroglitazar: copovidone is 1:2 and the premix having PXRD pattern as per FIG. 36;
c) Ratio of Saroglitazar magnesium: HPMC is 1:2, 1:3, 1:4, 1:5 and the premixes having PXRD patterns as per FIGS. 37, 38, 39, 40 respectively;
d) Ratio of Saroglitazar magnesium: Methacrylic acid is 1:2, 1:3, 1:4. 1:5 and the premixes having PXRD pattern as per FIGS. 41, 42, 43, 44 respectively;
e) Ratio of Saroglitazar: metformin is 1:1 and premix having PXRD pattern as per FIG. 45.

27. Pharmaceutical composition comprising compound claimed in claims and 1, 8, 15, 19 and 24 with suitable pharmaceutical excipients.
